(19) United States
(12) Patent Application Publication
(10) Pub. No.: US 2016/0101056 A1
(43) Pub. Date: Apr. 14, 2016

(54) MUCOADHESIVE DEVICES FOR DELIVERY OF ACTIVE AGENTS

(71) Applicants: ENTREGA INC., BOSTON, MA (US); THE REGENTS OF THE UNIVERSITY OF CALIFORNIA, OAKLAND, CA (US)

(72) Inventors: Baruch Z. HARRIS, Somerville, MA (US); Vijay M. K. MURTHY, Somerville, MA (US); David Adam STEINBERG, Milton, MA (US); Rodney PEARLMAN, El Granada, CA (US); Howard Brian ROSEN, Woodside, CA (US); Robert Samuel LANGER, Newton, MA (US); Hila ESPTEIN-BARASH, Newton, MA (US); Jonathan Robert BEHR, Cambridge, MA (US); Samir Suresh MITRAGOTRI, Santa Barbara, CA (US)

(21) Appl. No.: 14/867,229

(22) Filed: Sep. 28, 2015

Related U.S. Application Data
(63) Continuation of application No. 14/407,220, filed on Dec. 11, 2014, now abandoned.
(60) Provisional application No. 61/671,287, filed on Jul. 13, 2012.
(60) Provisional application No. 61/659,781, filed on Jun. 14, 2012.
(60) Provisional application No. 61/659,784, filed on Jun. 14, 2012.

(51) Int. Cl. A61K 9/20; A61K 38/28

(52) U.S. Cl. CPC A61K 9/2054; A61K 38/28

(57) ABSTRACT

Described herein are systems and methods for transmucosal delivery of active agents. In some embodiments, a system may comprise one or more mucoadhesive devices configured for release of an active agent.

### CROSS REFERENCE TO RELATED APPLICATIONS

This application claims the benefit of U.S. Provisional Application No. 61/659,784, filed Jun. 14, 2012. U.S. Provisional Application No. 61/659,781, filed Jun. 14, 2012, and U.S. Provisional Application No. 61/671,287, filed Jul. 13, 2012, each of the foregoing applications is incorporated by reference in its entirety.

### BACKGROUND

Oral dosing of active agents is attractive for many reasons, including ease of administration and high patient compliance. However, for some active agents, such as poorly absorbed, sensitive (i.e., pH sensitive, enzyme-sensitive, and the like.), and/or high molecular weight active agents, oral dosing may be less effective or ineffective for achieving sufficient blood concentration of the active agent as compared to alternative dosing strategies. For example, active agents such as proteins and other macromolecules may be enzymatically degraded in the gastrointestinal tract and/or may have limited transport across the intestinal epithelium.

One potential strategy for circumventing the hostile environment of the gastrointestinal tract is to alter the environment through the use of protease inhibitors and/or derivatization of active agents with polyethylene glycol to prevent enzymatic degradation. Another potential strategy is to increase the permeability of the tissue in the gastrointestinal tract such that absorption of an active agent increases. An active agent may be formulated with an excipient that can, for example, open the tight junctions of the intestine to allow an active agent to pass through the intestinal epithelium. A further approach to improving delivery of an active agent in the gastrointestinal tract is to apply an enteric coating to the active agent such that the active agent is released in the lower gastrointestinal tract where absorption of proteins occurs more readily.

Several modifications of simple dosage systems including liposomes, microparticles, and nanoparticles have been used as active agent carriers to overcome poor active agent bioavailability. For example, active agent-loaded mucoadhesive micro- and nanoparticles that adhere to intestinal mucus have been used to prolong the migration time of the particles in the intestine and extend release of the drug. However, several issues limit applicability of this approach. For example, the particles release active agent non-directionally, which results in active agent being lost to the lumen. Additionally, as a result of the surface of the particles being exposed to the intestinal fluid, active agents encapsulated in the particles may not be sufficiently protected from proteolytic degradation in the intestine.

### SUMMARY

Described herein are devices, systems and methods for transmucosal delivery of active agents. Exemplary transmucosal delivery methods include, e.g., oral transmucosal or intestinal transmucosal delivery.

In one aspect, a polymeric controlled release preparation is provided. The preparation comprises a mucoadhesive layer comprising a first region and a second region, the first region being substantially surrounded by the second region, wherein the first region comprises an active agent and the second region comprises a mucoadhesive material.

In another aspect, a pharmaceutically acceptable polymeric controlled release device for oral drug delivery is provided. The device comprises a mucoadhesive coating and a polymeric release layer comprising an active agent, wherein the polymeric release layer is disposed on the mucoadhesive coating; and wherein the mucoadhesive coating is capable of adhering to a mucosa with a force between 2 and 30 times the weight of the device.

In yet another aspect, a polymeric controlled release device is provided. The polymeric controlled release device comprises an active agent, a mucoadhesive layer, and a permeation enhancer, wherein when a surface of the device adheres to a mucosa defining a privileged region, a substantial majority of the permeation enhancer is maintained within the privileged region.

In still another aspect, a polymeric controlled release device is provided. The polymeric controlled release device comprises polymeric microparticles comprising an active agent. The polymeric controlled release device further comprises a mucoadhesive layer and a permeation enhancer, wherein when a surface of the device adheres to a mucosa in a lumen, a seal forms between the surface of the device and the mucosa defining a permeation region isolated from the lumen that at least partially prevents infiltration of components from the lumen into the permeation region.

In yet another aspect, a process for manufacturing a device as described above is provided. The process comprises applying a mucoadhesive coating to a wafer and compressing the mucoadhesive coating.

In still another aspect, a process for manufacturing a device as described above is provided. The process comprises providing a wafer having a first side and a second side, positioning the wafer on a surface where the first side of the wafer is in contact with the surface, applying a coating to the back side of the wafer to form the coated device, and removing the coated device from the surface, wherein the first side of the device is essentially free of the coating.

Also provided herein are systems for delivery of the mucoadhesive devices described herein. In some embodiments, a system may comprise one or more mucoadhesive devices configured for release of an active agent.

In one aspect an oral drug delivery system is provided. The system comprises a pharmaceutically acceptable containment vehicle comprising a plurality of polymeric devices configured for release in a controlled manner, wherein the devices each comprise a degradable mucoadhesive layer comprising a mixture of an active agent, a polymeric material, and a mucoadhesive material, wherein the mucoadhesive layer is configured to retain mucoadhesive properties during degradation of the mucoadhesive layer.

In another aspect, an oral drug delivery system is provided. The system comprises a pharmaceutically acceptable containment vehicle comprising a plurality of polymeric devices configured for release in a controlled manner, wherein the devices each comprise a polymeric layer comprising an active agent and a mucoadhesive coating configured to at least partially coat the surface of the polymeric layer.

In still another aspect, an oral drug delivery system is provided. The system comprises a pharmaceutically acceptable containment vehicle comprising a plurality of polymeric controlled release devices, wherein the controlled release devices each comprise an active agent and a mucoadhesive coating configured to partially coat the surface of the device, wherein the devices are configured to disperse upon release from the containment vehicle.

In yet another aspect, a first pharmaceutically acceptable polymeric controlled release device for oral drug delivery is provided. The device comprises a sacrificial layer, a mucoadhesive layer, and a polymeric release layer comprising an active agent, wherein the polymeric release layer is disposed on the mucoadhesive coating.

In still another aspect, a first pharmaceutically acceptable polymeric controlled release device for oral drug delivery is provided. The device comprises a sacrificial layer and a mucoadhesive layer comprising an active agent.

In yet another aspect, a method of orally delivering an active agent sensitive to degradation in the stomach is provided. The method comprises administering a system or device of any of the above claims to a patient in need thereof and informing the patient that the administration of the system or device during, immediately after, or within between about 30 minutes and 120 minutes of food intake results in an increase in device adhesion to small intestine compared to the administration without food.

In still another aspect, a method of orally delivering an active agent sensitive to degradation in the stomach is provided. The method comprises administering to a subject in need thereof a peristalsis activating agent and a system or device described above.

### BRIEF DESCRIPTION OF THE FIGURES

FIG. 1 shows a schematic of a device, according to an embodiment;

FIG. 2 shows a plot of active agent release as a function of time, according to an embodiment;

FIG. 3 shows a plot of percent active agent released as a function of time, according to an embodiment;

FIG. 4 shows a plot of cumulative insulin released as a function of time and is dose-proportional, according to an embodiment;

FIG. 5 shows a plot of cumulative insulin released as a function of time and is dose-independent, according to an embodiment;

FIG. 6 shows a intestinal fragments and a schematic for performing adhesive force tests, according to an embodiment;

FIG. 7 shows a bar graph of device adhesive force under various conditions, according to an embodiment;

FIG. 8 shows a bar graph of device detachment force as a function of pre-incubation time, according to an embodiment;

FIG. 9 shows a bar graph of device adhesion force as a function of time, according to an embodiment;

FIG. 10 shows a bar graph of device adhesive force under various conditions, according to an embodiment;

FIG. 11 shows a bar graph of device adhesive force under various conditions, according to an embodiment;

FIG. 12 shows a bar graph of device adhesive force relative to COSMOS-2.0 of various COSMOS materials, according to an embodiment;

FIG. 13 shows a bar graph of device adhesive force of various COSMOS materials, according to an embodiment;

FIG. 14 shows a bar graph of fold reduction in release for various device configurations, according to an embodiment;

FIG. 15 shows a bar graph of FITC-insulin transported as a function FITC-insulin per well, according to an embodiment;

FIG. 16 shows a bar graph of percent FITC-insulin transported for various device configurations according to an embodiment;

FIG. 17 shows a bar graph of percent rhodamine transported for various device configurations, according to an embodiment;

FIG. 18 shows a bar graph of percent cell survival for various device materials, according to an embodiment;

FIG. 19 shows a plot of insulin released as a function of time from devices containing various additives, according to an embodiment;

FIG. 20 shows three bar graphs of percent active agent released from devices that do not contain Tween-20 versus devices that contain Tween-20, according to an embodiment;

FIG. 21 shows a bar graph of device adhesive force for devices containing Tween-20 or various active agents, according to an embodiment;

FIG. 22 shows two plots showing the effect of device geometry and a permeation enhancer on blood glucose levels (left plot) and plasma insulin levels (right plot) as a function of time in rats, according to an embodiment;

FIG. 23 shows in-vitro calcitonin release of 24 μg calcitonin per 5 mm mucoadhesive device in PBS (pH 7.4, data represent mean±SE), according to an embodiment;

FIG. 24 shows wafer-assisted calcitonin transport across caco-2 monolayers (data represent mean±SE), according to an embodiment; and

FIG. 25 shows pharmacodynamic profiles of calcitonin (reduction in plasma calcium concentration) following placement of calcitonin wafers in the duodenum and jejunum in the rat small intestine (data represent mean±SE), according to an embodiment.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention.

### DETAILED DESCRIPTION

Described herein are devices, systems, and methods for transmucosal, oral, and/or transintestinal delivery of active agents. In some embodiments, a system may comprise one or more mucoadhesive devices (e.g., wafers) configured for release of an active agent. In one aspect, mucoadhesive devices for delivery of active agents are provided, In some cases, the device may be at least partially encapsulated by a backing layer having minimal permeability to the active agent (e.g., an active pharmaceutical ingredient). The device may, in some embodiments, comprise a plurality of layers. For example, the device may comprise a mucoadhesive layer and polymeric layer comprising an active agent. In other embodiments, the device may comprise a mucoadhesive layer comprising an active agent. In some embodiments, the device may adhere to a mucosa with a force of at least about 0.5, at least about 1, at least about 1.5, at least about 2, at least about 10, at least about 50, or at least about 100 times the weight of the device. Other aspects described herein are directed to methods of administerin the devices and enhancing transmucosal delivery of active agents.

Advantageously, the device may allow directional (e.g., unidirectional) delivery of active agents (e.g., active pharmaceutical ingredients) to a tissue (e.g., a mucosa) for absorption by a subject. Surprisingly, the inventors have found that directional delivery of active agents can dramatically increase the local concentration of an active agent, thereby potentially improving the absorption of the active agent by the subject. In some embodiments, active agents that have previously been difficult or impossible to deliver orally can be delivered using the systems and devices contemplated herein. For example, in some embodiments, the active agent may be sensitive to degradation in the stomach of a non-human animal. In another example, the active agent may be sensitive to degradation in the stomach of a human. Without wishing to be bound by any theory, it is believed that the device may allow an active agent to be absorbed by the gastrointestinal system of a subject without the need of a cellular receptor and/or without disruption of a tight junction. In some embodiments, the inclusion of an optional backing layer on the device may facilitate directional delivery of an active agent by limiting or preventing elution of the active agent from regions of the device other than the intended elution surface (e.g., the attachment surface). Also advantageously, the device may allow a desired active agent to be absorbed by a subject while substantially preventing absorption of undesired molecules.

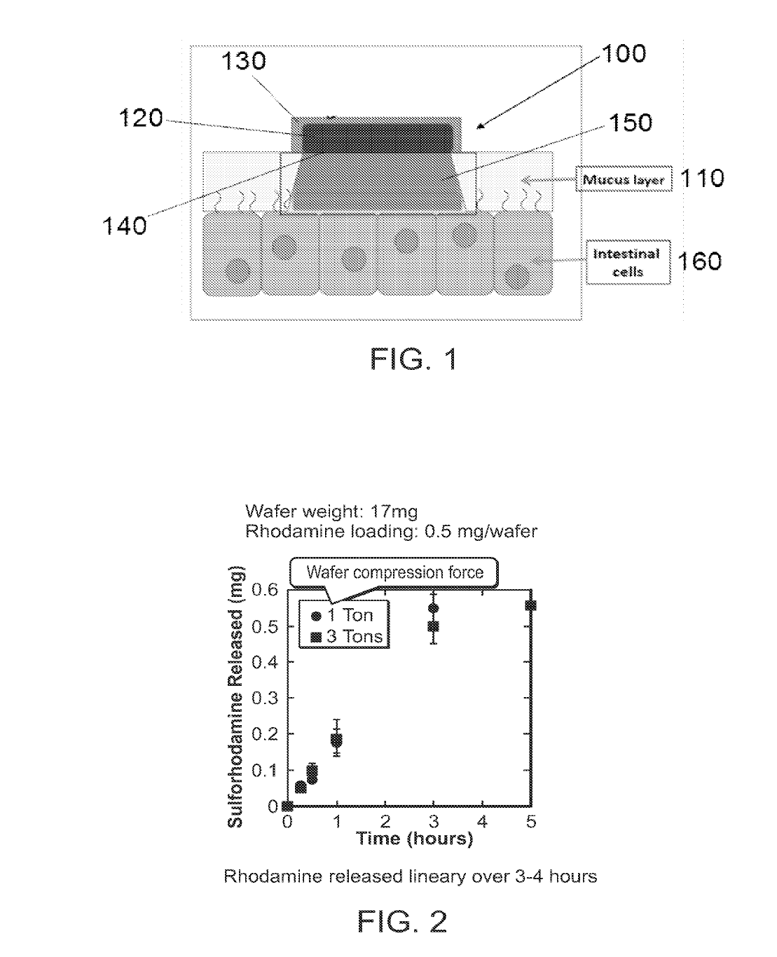

Referring now to FIG. 1, a non-limiting example of a device 100 is shown adhered to the mucosa 110 of an intestine. Device 100 comprises an active agent release compartment 120 and a backing layer 130 that covers at least some of the active agent release compartment but not the attachment surface 140 of the active agent release compartment. The optional backing layer 130 has limited permeability to the active agent such that release of the active agent is directed towards the mucosa resulting in an enhanced local concentration 150 of the active agent (e.g., active pharmaceutical ingredient). The active agent may pass through or between intestinal cells 160 to enter the circulation system of the subject.

In some embodiments, the active agent release compartment may comprise a mucoadhesive material. The inucoadliesive material may be any suitable, biocompatible mucoadhesive material. In some cases, the mucoadhesive material may be degradable or nondegradable. The mucoadhesive material may comprise, in some embodiments, a polymer (i.e., a natural or synthetic polymer). In some embodiments, the mucoadhesive material may comprise a Carbopol polymer [e.g, Carbopol 934 (BF Goodrich Co., Cleveland, Ohio)], carbomer, polycarbophil, pectin, a modified cellulose (e.g., caboxymethyl cellulose, sodium carboxymethylcellulose, hydroxymethyl propyl cellulose, hydroxypropyl methylcellulose, hydroxyethyl cellulose, hydroxypropyl cellulose, and the like), polyanhydrides, polymers and copolymers of acrylic acid, methactylic acid, and their lower alkyl esters [e.g., polyacrylic acid, poly(methyl methacrylates), poly(ethyl methacrylates), polybutylmethacrylate), polyisobutyl methacrylate), poly(hexylmethacrylate), poly(isodecyl methacrylate), poly(lauryl methacrylate), polyphenyl methacrylate), poly(methyl acrylate), poly(isopropyl acrylate), polyisobutyl acrylate), and poly(octadecyl acrylate)], polyvinyl alcohol, sodium hyaluronate, polyvinylpyrrolidone (PVP), chitosan, zinc-pectinate chitosan, calcium-chaitosan, chemically modified chitosan such as thiolated chitosan, alginate, xanthum gun, Fructooligosaccharides (FOS), glucose, dextran, and copolymers and blends thereof. In some embodiments, the mucoadhesive material may comprise a mixture of two or more materials (e.g., polymers).

In some embodiments, the mucoadhesive material may be dispersed throughout the active agent release compartment. In some instances, the mucoadhesive material may be present in a first region of the device but essentially not present in a second region of the device. For example, in some cases, the device may comprise a mucoadhesive layer comprising the mucoadhesive material.

In some cases, the active agent release compartment may comprise a pharmaceutically acceptable canter. For example, in some embodiments, the active agent release compartment may comprise one or more excipients, solubilizers, plasticizers, crystallization inhibitors, bulk filling agents, bioavailability enhancers, and combinations thereof. In some embodiments, the active agent release compartment may comprise polyethylene glycols, humectants, vegetable oils, medium chain mono, di and triglycerides, lecithin, waxes, hydrogenated vegetable oils, colloidal silicon dioxide, polyvinylpyrrolidone (PVP) (“povidone”), celluloses. CARBOPOL® polymers (Lubrizol Advanced Materials, Inc.) (i.e., crosslinked acrylic acid-based polymers), acrylate polymers, or other hydrogel forming polymers. Suitable pharmaceutically acceptable carriers may be determined based on factors including, but not limited to, the dosage form, desired release rate of the drug, and stability of the drug to be delivered.

In some cases, the active agent release compartment may comprise a material that retards or enhances the rate of release of the active agent (e.g., a release-rate control excipient). In some embodiments, the release-rate control excipient may enhance the release rate of the active agent as compared to a device without the release-rate control excipient. In some cases, the release-rate control excipient may be a surfactant (e.g., Tween or pyridinium propyl sulfonate). In other embodiments, the release-rate control excipient may be a high ionic strength material (e.g., sodium chloride). Without wishing to be bound by any theory, the release-rate control excipient may increase the rate of active agent release by decreasing the affinity of the active agent for a material in the active agent release compartment (e.g., a polymer). Thus, in some instances, the release-rate control excipient may be any material capable of decreasing the affinity of the active agent for a material in the active agent release compartment.

In other embodiments, the release-rate control excipient may be any material capable of blocking an active agent from binding to a material in the active agent release compartment. In one non-limiting example, a protein (e.g., bovine serum albumin) that may be capable of binding to a material in the active agent release compartment may be combined with an active agent in an amount sufficient to block the active agent from binding to the material in the active agent release compartment, thus facilitating release of the active agent from the device.

In certain embodiments, the release-rate control excipient may be a water soluble leachable material (e.g, a salt such as sodium chloride, a sugar such as sucrose, and the like). In some embodiments, the leachable material may dissolve when the device is in contact with an aqueous solution, thereby creating channels within the device and increasing the rate of release of the active agent.

In some embodiments, the active agent release compartment may comprise a release rate-modifying polymer. The release rate-modifying polymer may be degradable or nondegradable. The release rate-modifying polymer may, in some embodiments, be a natural or synthetic polymer. In certain embodiments, the release rate-modifying polymer may comprise poly(hydroxy acids) [e.g., poly(lactic acid), poly(glycolic acid), and polylactic acid-co-glycolic acid)], polyanhydrides, polyorthoesters, polyamides, polycarbonates, polyalkylenes (e.g., polyethylene and polypropylene), polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, polyvinyl alcohols, poly(vinyl acetate), polystyrene, polyurethanes, derivatized celluloses (e.g., alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulfate sodium salt (jointly referred to herein as “synthetic celluloses”)], polymers of acrylic acid, methacrylic acid or copolymers or derivatives thereof including esters, poly(methyl methacrylate), poly(ethyl methacrylate), polybutylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), and copolymers and blends thereof. Examples of non-biodegradable polymers include ethylene vinyl acetate, poly(meth)acrylic acid, polyamides, copolymers and blends thereof. Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

In some embodiments, the device comprises a backing layer. The backing layer may have limited permeability and/or may be impermeable to, e.g., certain biological agents. In some embodiments, the backing layer may be selectively permeable. For example, in some embodiments, the backing layer may be permeable to fluid and impermeable to certain solutes dissolved in the fluid (e.g, proteins). In some embodiments, the backing layer may at least partially prevent leakage of the active agent from the active agent release compartment. In some instances, the backing layer may at least partially prevent infiltration of undesirable molecules into the active agent release compartment. In some embodiments, the backing layer may be essentially non-mucoadhesive. In other embodiments, the backing layer may have reduced mucoadhesiveness relative to the attachment surface of the device, as described in more detail elsewhere herein. In some embodiments, the backing layer may be dearadable. In some instances, the backing layer may be more slowly degradable than other components of the device. In certain embodiments, the backing layer may substantially protect the device's overall integrity after administration.

The backing layer may comprise any suitable, biocompatible material. In some cases, the backing layer may be degradable or nondegradable. The backing layer may comprise, in some embodiments, a polymer (i.e., a natural or synthetic polymer). In some embodiments, the backing layer may comprise poly(hydroxy acids) [e.g., poly(lactie acid), poly(glycolic acid), and polylactic acid-co-glycolic acid)], polyanhydrides, polyorthoesters, polyamides, polycarbonates, polyalkylenes (e.g., polyethylene and polypropylene), polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, polyvinyl alcohols, poly(vinyl acetate), polystyrene, polyurethanes, derivatized celluloses (e.g., alkyl cellulose, hydroxyalkyl cellulose, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxyethyl cellulose, cellulose triacetate, and cellulose sulfate sodium salt (jointly referred to herein as “synthetic celluloses”)], polymers of acrylic acid, methacrylic acid or copolymers or derivatives thereof including esters, poly(methyl methacrylate), poly(ethyl methacrylate), polybutylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), and copolymers and blends thereof. Examples of non-biodegradable polymers include ethylene vinyl acetate, poly(meth)acrylic acid, polyamides, copolymers and blends thereof. Examples of biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Any suitable polymer can be used in accordance with the devices and/or systems described herein. Such polymers can be natural or unnatural synthetic) polymers. Polymers can be homopolymers or copolymers comprising two or more monomers. In terms of sequence, copolymers can be random, block, or comprise a combination of random and block sequences. Contemplated polymers may be biocompatible and/or biodegradable. The polymers described herein (for any purpose) may have any suitable molecular weight. In some embodiments, a polymer may have a molecular weight of between about 10 kDa to about 2000 kDa, in some embodiments between about 20 kDa and about 1000 kDa, in some embodiments between about 40 kDa and about 1000 kDa, and in some embodiments, between about 40 kDa and about 500 kDa.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., below concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, PLA, polycaprolactone, or copolymers or derivatives including these and/or other polymers.

In certain embodiments, biocompatible polymers may be biodegradable, i.e., the polymer is able to degrade, chemically and/or biologically, within a physiological environment, such as within the body. As used herein, “biodegradable” polymers are those that, when introduced into the body of a subject, are broken down by the cellular machinery or excreted products (i.e., biologically degradable) and/or by a chemical process, such as hydrolysis (e.g., chemically degradable) into components that the body can either reuse or dispose of without significant toxic effect. In one embodiment, the biodegradable polymer and their degradation byproducts can be biocompatible.

For instance, a polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form actic acid, polyglycolide may be hydrolyzed to form glycolic acid, etc.).

In some embodiments, polymers may be polyesters, including copolymers comprising lactic acid and glycolic acid units, such as poly(lactic)-co-poly(glycolic) acid, poly(lactic acid-co-glycolic acid), and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids or polyanhydrides.

In other embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylatc copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

PLGA contemplated for use as described herein can be characterized by a lactic acid:glycolic acid ratio of e.g., approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., a PLGA block copolymer or PLGA-PEG block copolymer), may be selected to optimize for various parameters such as water uptake, therapeutic agent release and/or polymer degradation kinetics can be optimized. In other embodiments, the end group of a PLA polymer chain may be a carboxylic acid group, an amine group, or a capped end group with e.g., a long chain alkyl group or cholesterol. Devices disclosed herein may or may not contain polyethylene glycol.

In some embodiments, one or more plasticizers may be added to the backing layer. In some embodiments, the plasticizer may facilitate compliance of the backing layer with swelling of the device. Representative classes of plasticizers include, but are not limited to, abietates, adipates, alkyl sulfonates, azelates, benzoates, chlorinated paraffins, citrates, energetic plasticizers, epoxides, glycol ethers and their esters, glutarates, hydrocarbon oils, isobutyrates, oleates, pentaerythritol derivatives, phosphates, phthalates, polymeric plasticizers, esters, polybutenes, ricinoleates, sebacates, sulfonamides, tri- and pyromellitates, biphenyl derivatives, calcium stearate, carbon dioxide, difuran diesters, fluorine-containing plasticizers, hydroxybenzoic acid esters, isocyanate adducts, multi-ring aromatic compounds, natural product derivatives, nitrites, siloxane-based plasticizers, tar-based products and thioesters. An exemplary plasticizer is glycerol at a concentration of about 2% w/v.

In some embodiments, the backing layer may comprise one or more layers. For example, in some instances, the backing layer may comprise a first layer that has limited permeability to a first class of molecules and a second layer that has limited permeability to a second class of molecules. The permeability of the backing layer may, in some cases, be related to one or more properties of the molecules for which it has limited permeability. For example, the molecular weight and/or charge of a molecule may influence the permeability of the molecule through the backing layer. In some embodiments, the backing layer may be essentially impermeable to molecules having a molecular weight above about 50 Da, in some embodiments above about 100 Da, in some embodiments above about 200 Da, in some embodiments above about 300 Da, in some embodiments above about 500 Da, in some embodiments above about 1000 Da, in some embodiments above about 2000 Da, in some embodiments above about 5000 Da, and in somc embodiments above about 10000 Da.

In some embodiments, the device may comprise a sacrificial layer that essentially prevents or substantially reduces release of the active agent. For example, in some embodiments, the sacrificial layer may coat the attachment surface of the device. In some embodiments, the sacrificial layer may coat substantially the whole device. The sacrificial layer may comprise any suitable material that can prevent or reduce release of the active agent when such properties are desirable and can allow or increase release of the active agent when such properties are desirable. In some embodiments, the release-altering properties of the sacrificial layer decrease as the sacrificial layer dissolves or degrades. Thus, in some embodiments, the sacrificial layer may be any suitable material that performs as described above. In some embodiments, the sacrificial layer may be a polymet (e.g., a degtadable nondegiadable polymer).

Any of the layers described herein may be any suitable thickness. For example, in some embodiments, a layer may be less than about 10 mm thick, in some embodiments less than about 5 mm thick, in some embodiments less than about 1 mm thick, in some embodiments less than about 500 microns thick, in some embodiments less than about 200 microns thick, in sonic embodiments less than about 100 microns thick, in some embodiments less than about 50 microns thick, in some embodiments less than about 20 microns thick, in some embodiments less than about 10 microns thick, in some embodiments less than about 5 microns thick, in some embodiments less than about 2 microns thick, in some embodiments less than about 1 micron thick, in some embodiments less than about 500 nm thick, in some embodiments less than about 200 nm thick, in some embodiments less than about 100 nm thick, and in some embodiments less than about 50 nm thick. In certain embodiments, a layer may be between about 50 nm thick and about 10 mm thick, in some embodiments between about 50 nm thick and about 1 mm thick, in some embodiments between about 500 nm thick and about 10 mm thick, in some embodiments between about 500 nm thick and about 1 mm thick, in some embodiments between about 1 micron thick and about 1 mm thick, in some embodiments between about 1 micron thick and about 100 microns thick, in some embodiments between about 1 micron thick and about 10 microns thick, in some embodiments between about 10 microns thick and about 1 mm thick, and in some embodiments between about 50 nm thick and about 10 microns thick. In other embodiments, a layer may be at least about 1 micron thick, in some embodiments at least about 10 microns thick, in some embodiments at least about 100 microns thick, in some embodiments at least about 1 mm thick, and in some embodiments at least about 10 mm thick.

In some embodiments, the device may comprise an active agent. In some cases, the device may comprise two or more active agents. In some instances, the active agent may be a peptide, a protein, a nucleic acid, a polysaccharide, a small inorganic molecule, or a small organic molecule. A wide range of active agents may be included in the compositions. The active agents may include alternative forms such as alternative salt forms, free acid forms, free base forms, and hydrates. In some embodiments, an active agent may be selected from a list of known agents, for example, a list of agents previously synthesized, a list of agents previously administered to a subject, for example, a human subject or a mammalian subject, a list of FDA approved agents, or a historical list of agents, for example, a historical list of a pharmaceutical company, etc. Suitable lists of known agents are well known to those of skill in the art and include, but are not limited to, the Merck Index and the FDA Orange Book, each of which is incorporated herein by reference. In some cases, small molecules and libraries of small molecules can be obtained from commercial and academic sources, for example, from Sigma-Aldrich (www.sigmaaldrich.com), ChemDiv (www.chemdiv.com), Evotec (www.evotec.com), or ICCB (iccb.med.harvard.edu/screening/compound_libraries/index.htm). In some embodiments, active agents that are not amenable to conventional oral administration may be successfully administered using the devices contemplated herein. For example, in some instances, an active agent that may be rendered ineffective by enzymatic degradation following conventional oral administration may be successfully administered using the devices contemplated herein. In some embodiments, the contemplated devices may be used to administer to a subject an active agent that may be sensitive to the gastrointestinal system environment.

An active agent may be included in the device in any suitable amount or concentration. For example, the active agent may be at least about 1% of the weight of the device, in some embodiments at least about 2% of the weight of the device, in some embodiments at least about 3% of the weight of the device, in some embodiments at least about 4% of the weight of the device, in some embodiments at least about 5% of the weight of the device, in some embodiments at least about 10% of the weight of the device, in some embodiments at least about 15% of the weight of the device, in some embodiments at least about 20% of the weight of the device, in some embodiments at least about 25% of the weight of the device, in some embodiments at least about 30% of the weight of the device, in some embodiments at least about 35% of the weight of the device, in some embodiments at least about 40% of the weight of the device, in some embodiments at least about 45% of the weight of the device, and in some embodiments at least about 50% of the weight of the device. In certain embodiments, the active agent may be between about 1% and about 50% of the weight of the device, in some embodiments between about 1% and about 40% of the weight of the device, in some embodiments between about 1% and about 30% of the weight of the device, in some embodiments between about 2% and about 50% of the weight of the device, and in some embodiments between about 5% and about 50% of the weight of the device. It should be understood that the active agent may comprise one or more compounds.

In some embodiments, the device may be configured for controlled release of an active agent. In other embodiments, the device may be configured for a burst release of an active agent. In some instances, two or more active agents may be located in different regions of the device. In some embodiments, the two or more active agents may each be different from each other or at least some of the two or more active agents may be the same. Such configurations may be advantageous, for example, for controlling the release rate of an active agent. For instance, a first active agent in a first region may be configured to release at a first rate and a second active agent in a second region may be configured to release at a second rate, where the first rate and the second rate are different. For example, the first rate may be higher (e.g., a burst release) than the second rate (e.g., a sustained release). A burst release refers to a much higher release rate during a first period of time as compared to a second period of time. In some embodiments, the burst release may occur during the initial period of drug release, i.e., beginning when the device is placed in an environment in which drug release can occur. In other embodiments, the burst release may occur after a period of sustained (e.g., zero-order) release. Various release rates are discussed in more detail elsewhere herein. An initial burst release may be advantageous, in some embodiments, for rapidly achieving a desired blood concentration of the active agent in a subject after which a slower sustained release of the active agent may be used to maintain a desired blood concentration of the active agent. Of course, the first active agent the second active agent may be the same or different. In another embodiment, two or more formulations of one or more active agents may be included in the device. The formulations may be contained in separate regions or may exist as a mixture. In some embodiments, a first formulation may have a first rate of release and the sccond formulation may have a second rate of release, where the first rate of release is different from the second rate of release.

In some embodiments, the device may comprise a mucoadhesive material, an active agent release compartment comprising an active agent, and a backing layer, where the mucoadhesive material is dispersed throughout the active agent release compartment. In some embodiments, the active agent release compartment may comprise a plurality of layers. For example, the active agent release compartment may comprise a release layer comprising the active agent and a mucoadhesive layer.

In some embodiments, the device may comprise a release layer that modulates the release of an active agent from the device. The release layer may, in some embodiments, be disposed on the mucoadhesive layer. In some cases, the release layer may be polymeric. In some instances, the release layer may be degradable or nondegradable. In certain embodiments, the release layer may have pores through which active agent can elute.

In some embodiments, the device may comprise a plurality of microparticles and/or nanoparticles for controlled release of the active agent. The particles may, in some embodiments, be formed from one or more polymers, such as those described herein. In some cases, the particles may be dispersed throughout the active agent release compartment. In other embodiments, the particles may be located in a particular region of the active agent release compartment.

The devices described herein may have any suitable dimensions. For example, in some embodiments, the devices may be cylindrical or spherical. In some instances, the device may have a dimension greater than about 1 micron, in some embodiments greater than about 5 microns, in some embodiments greater than about 10 microns, in some embodiments greater than about 20 microns, in some embodiments greater than about 50 microns, in some embodiments greater than about 100 microns, in some embodiments greater than about 200 microns, in some embodiments greater than about 500 microns, in some embodiments greater than about 1 mm, in some embodiments greater than about 5 mm, and in some embodiments greater than about 10 mm. In some embodiments, the device may have a dimension between about 1 nm and about 5 mm, in some embodiments between about 1 nm and about 1 mm, in some embodiments between about 10 nm and about 1 mm, in some embodiments between about 100 nm and about 1 mm, in some embodiments between about 1 micron and about 1 mm, in some embodiments between about 10 microns and about 1 mm, and in some embodiments between about 100 microns and about 5 mm.

In some embodiments, the device may be sufficiently flexible such that it is capable of flexing to substantially accommodate a curved surface. For example, the device may be capable of accommodating an uneven surface of a tissue (e.g., a mucosa).

In some instances, a plurality of devices may be provided. In some embodiments, the plurality of devices may be substantially the same. In other embodiments, the plurality of devices may be substantially different. In certain embodiments, a portion of a plurality of devices may be substantially the same and a portion of the plurality of devices may be substantially different.

In some embodiments, the rate of active agent release from the device may be at least about 0.001 micrograms per hour, in some embodiments at least about 0.005 micrograms per hour, in some embodiments at least about 0.01 micrograms per hour, in some embodiments at least about 0.05 micrograms per hour, in some embodiments at least about 0.1 micrograms per hour, in some embodiments at least about 0.5 micrograms per hour, in some embodiments at least about 1 microgram per hour, in some embodiments at least about 5 micrograms per hour, in some embodiments at least about 10 micrograms per hour, in some embodiments at least about 20 micrograms per hour, in some embodiments at least about 50 micrograms per hour, in some embodiments at least about 100 micrograms per hour, in some embodiments at least about 500 micrograms per hour, in some embodiments at least about 1 mg per hour, in some embodiments at least about 5 mg per hour, or even more. It should be understood that a device may release an active agent at any of these rates even if releasing the drug for less than about 1 hour. In some embodiments, the rate of active agent release may be essentially independent of the active agent loading of the device. In some embodiments, the cumulative amount of active agent released may increase directly as a function of the active agent loading of the device.

In certain embodiments, the active agent may be released in an essentially linear fashion. For example, the release may be essentially zero-order. In some cases, the release may be non-linear. The release may, in some instances, be non-linear for a first period of time and essentially linear for a second period of time.

In some embodiments, at least a portion of the device may swell as a function of pH. For example, the region containing the active agent may swell when exposed to an aqueous environment having a pH of between about 1 and about 3, in some embodiments between about 3 and about 5, in some embodiments between about 5 and about, in some embodiments between about 7 and about 9, in some embodiments between about 1 and about 5, in some embodiments between about 3 and about 7, in sonic embodiments between about 5 and about 9, and in some embodiments between about 6 and about 8. In some instances, the rate of active agent release is modulated by the swelling of the device. For example, in some instances, the active agent release rate may increase upon swelling of the device. In other instances, the active agent release may decrease upon swelling of the device.

In some embodiments, the attachment surface of a device may regenerate its mucoadhesiveness essentially continuously. For example, the mucoadhesive material of a device may continuously elute, degrade, erode, and/or dissolve. Thus, in some embodiments, the device may retain at least some of its mucoadhesive properties even as at least some of the mucoadhesive material is lost, neutralized, or otherwise rendered less effective or substantially ineffective.

In some embodiments, the backing layer and the active agent release compartment may each swell at a rate relative to each other sufficient to maintain the integrity of the device. In some embodiments, the backing layer and/or the active agent release compartment [and/or one or more layers the backing layer and/or active agent release compartment (e.g., the mucoadhesive layer)] should be sufficiently flexible such that when the device swells due (e.g., due to water absorption), one or more layers do not separate (e.g., delaminate). As discussed herein, in some embodiments, the flexibility of a layer or region of the device may be modified by incorporation of one or more additives (e.g., a plasticizer) into the layer or region. In some embodiments, the active agent release compartment may swell at a first rate and the backing layer may swell at a second rate, where the first rate and the second rate differ by less than about 50%, in some embodiments less than about 20%, in some embodiments less than about 10%, in some embodiments less than about 5%, and in some embodiments less than about 1%.

In some cases, the device may adhere to a mucosa. For instance, the device may adhere to the mucosa of the small intestine (e.g., the duodenum, jejunum, or ileum) and/or the large intestine (e.g., the ascending colon, the right colic flexure, the transverse colon, the transverse mesocolon, the left colic flexure, the descending colon, the sigmoid colon, and the rectum). In some embodiments, the device may adhere to a mucosa anywhere between the pyloric sphincter and the rectum. In some embodiments, the device may be capable of inserting into an invagination of an intestinal membrane.

In certain embodiments, the device may adhere to a mucosa and remain adhered for a period of time. For example, in some instances, the device may adhere to a mucosa for less than about 7 days, in some embodiments for less than about 5 days, in some embodiments for less than about 2 days, in some embodiments for less than about 1 day, in some embodiments for less than about 12 hours, and in some embodiments less than 4 hours. In some cases, the device may adhere to a mucosa for between about 30 minutes and about 7 days, in some embodiments between about 30 minutes and about 2 days, in some embodiments between about 30 minutes and about 1 day, in some embodiments between about 30 minutes and about 12 hours, in some embodiments between about 30 minutes and about 4 hours, in some embodiments between about 1 hour and about 24 hours, in some embodiments between about 2 hours and about 12 hours, in some embodiments between about 2 hours and about 6 hours and in some embodiments between about 3 hours and about 4 hours. In certain embodiments, the device may adhere to a mucosa for at least about 30 minutes, in some embodiments at least about 1 hour, in some embodiments at least about 2 hours, in some embodiments at least about 3 hours, in some embodiments at least about 6 hours, in some embodiments at least about 12 hours, and in some embodiments at least about 1 day.

In some cases, a device having mucoadhesive properties for at least some of the time in the gastrointestinal system of a subject may travel though the gastrointestinal system of a subject at a slower rate than a device that is substantially free of mucoadhesive properties.

In some embodiments, the device may adhere to tissue (e.g., a mucosa) with significant force. For instance, in some cases, the device may adhere to tissue with a force greater than about 0.5, greater than about 1, greater than about 1.5, or greater than about 2 times the weight of the device, in some embodiments greater than about 5 times the weight of the device, in some embodiments greater than about 10 times the weight of the devicc in some embodiments greater than about 20 times the weight of the device, in some embodiments greater than about 50 times the weight of the device, and in some embodiments greater than about 100 times the weight of the device. In some embodiments, the device may adhere to tissue with a force between about 2 and about 20 times the weight of the device, in some embodiments between about 50 and about 100 times the weight of the device, and in some embodiments between about 100 and about 500 times the weight of the device. In some cases, the device may adhere to a tissue with a force of at least about 1 mN, in some embodiments at least about 2 mN, in some embodiments at least about 5 mN, in some embodiments at least about 10 mN, in some embodiments at least about 20 mN, in some embodiments at least about 50 mN, in some embodiments at least about 100 mN, in some embodiments at least about 200 mN, and in some embodiments at least about 500 mN.

In some embodiments, the adhesiveness of a device may be modulated by preincubation of the device in an aqueous solution. In some cases, the adhesiveness of a device may increase during preincubation such that the device may adhere to tissue with substantially greater initial force as compared to a device that had not been preincubated in aqueous solution. In some embodiments, the adhesiveness of a device may persist for a period of time after exposure to an aqueous environment but before adhesion of the device to tissue (e.g., a mucosa). In certain embodiments, the device may have greater adhesiveness when in contact with an intestinal fluid as comparcd to when in contact with a non-intestinal aqueous solution. In other embodiments, the device may have less adhesiveness when in contact with an intestinal fluid as compared to when in contact with a non-intestinal aqueous solution.

In some cases, the attachment surface of a device may be significantly more mucoadhesive than the backing layer of the device. In some embodiments, the backing layer may be essentially nonmucoadhesive.

In some embodiments, systems and devices embraced herein may be biocompatible. In certain embodiments, the systems and devices may be substantially inert to the immune systcm of a subject. In some cases, the systems and devices comprise materials that are considered safe and effective and may be administered to an individual without causing undesirable biological side effects or unwanted interactions. In certain embodiments, the device may have a minimal effect (e.g., histological effect) on the region of tissue where the device adheres. For example, the device, in some embodiments, may leave the region of tissue where it adheres essentially undamaged.

In some embodiments, the device may adhere to a tissue (e.g., a mucosa) and form a seal. For example, the device may adhere to a tissue and create a privileged region of the tissue that is at least partially isolated from the region of tissue outside of the seal. In some embodiments, the privileged region may have substantially improved permeation of the active agent relative to the region outside the privileged region. In some cases, substantially all of the privileged region of tissue is adhered to the device. In some embodiments, the attachment region of the device may comprise a first region having reduced or substantially no mucoadhesive properties and a second region having mucoadhesive properties. In some embodiments, the second region may comprise an active agent. In some embodiments, the first region may be substantially free of active agent. In certain embodiments, the attachment region may have a ring configuration where the first region is surrounded by the second region. In some embodiments, the first region may be an ellipsoid. In other embodiments, the first region may be a cylinder. In certain embodiments, first region may be round, oval, triangular, quadrangular polygonal, or irregular.

In certain embodiments, the seal formed by adhesion of the device to a tissue may be capable of limiting or even substantially excluding infiltration of molecules from outside the privileged region to the privileged region. This property may be advantageous, for example, in the intestine where it may be desirable to prevent intraluminal materials from entering the privileged region and being absorbed by the subject. In some embodiments, molecules substantially unable to infiltrate the privileged region may have a molecular weight greater than about 50 Da, in some embodiments greater than about 100 Da, in some embodiments greater than about 200 Da, in some embodiments greater than about 500 Da, in some embodiments greater than about 1000 Da, in some embodiments greater than about 2000 Da, in some embodiments greater than about 5000 Da, and in some embodiments greater than about 10000 Da. In certain embodiments, an active agent may be substantially prevented from escaping the privileged region to the intraluminal region.

In some embodiments, absorption of an active agent may be improved by a permeation enhancer. In some embodiments, the seal formed by the device may substantially prevent escape of the permeation enhanccr from the privileged region. In some embodiments, the permeation enhancer may be substantially retained in the privileged region. In some cases. the permeation enhancer may reduce the viscosity of mucus. In some embodiments, the permeation enhancer may be capable of opening a tight junction. A permeation enhancer may, in some instances, facilitate uptake of an active agent into epithelial cells.

Representative classes of permeation enhancers include, but are not limited to, a fatty acid, a medium chain glyceride, a surfactant, a steroidal detet gent, an acyl carnitine, lauroyl-DL-carnitine, alkanoyl choline, an N-acetylated amino acid, esters, salts, bile salts, sodium salts, nitrogen-containing rings, derivatives thereof, and combinations thereof. The enhancer can be anionic, cationic, zwitterionic, or nonionic, Anionic permeation enhancers can include, but are not limited to, sodium lauryl sulfate, sodium decyl sulfate, sodium octyl sulfate. N-lauryl sarcosinate, and sodium corparate. Cationic permeation enhancers can include, but arc not limited to, cetyltrimethyl ammonium bromide, decyltrimethyl ammonium bromide, benzyldimethyl dodecyl ammonium chloride, myristy ltimethyl ammonio chloride, and deodecyl pridinium chloride. Zwitterionic permeation enhancers can include, but are not mited to, decyldimethyl ammonio propane sulfonate, palmityldimethyl ammonio propane sulfonate. Fatty acids can include, but are not limited to, butyric, caproic, caprylic, pelargonic, capric, lauric, myristic, palmitic, stearic, arachidic, oleic, linoleic, and linolinic acid, salts thereof, derivatives thereof, and combinations thereof. In some embodiments, a fatty acid may be modified as an ester, for example, a glyceride, a monoglyceride, a diglyceride, or a triglyceride. Bile acids or salts including conjugated or unconjugated bile acid permeation enhancers can include, but are not limited to, cholate, deoxycholate, tauro-chotate, glycocholate, taurodexycholate, ursodeoxycholate, tauroursodeoxycholate, chenodeoxycholate, derivates thereof, salts thereof, and combinations thereof. In some embodiments, permeation enhancers can comprise a metal chelator, such as EDTA or EGTA, a surfactant such as sodium dodecyl sulfate, polyethylene ethers or esters, polyethylene glycol-12 lauryl ether, salicylate polysorbate 80, nonylphenoxypolyoxyethylene, dioctyl sodium sulfosuccinate, saponin, palmitoyl carnitine, lauroyl-1-camitine, dodecyl maltoside, acyl carnitines, alkanoyl cjolline, and combinations thereof. Other permeation enhancers can include, but are not limited to, 3-nitrobenzoate, zoonula occulden toxin, fatty acid ester of lactic acid salts, glycyrrhizic acid salt, hydroxyl beta-cyclodextrin, N-acetylated amino acids such as sodium N-[8-(2-hydroxybenzoyl)aminolcaprylate and chitosan, salts thereof, derivatives thereof, and combinations thereof. An exemplary permeation enhancer is 1% by weight palmityldimethyl ammonio propane sulfonate (PPS). Permeation enhancers are also described in Whitehead et al., J. Control. Release, 128 (2008) 128-133 and in Whitehead et al., Pharm. Res., 25 (2008) 1782-1788, the entire contents of which are incorporated herein by reference.

In some embodiments, a permeation enhancer may be included in a device (e.g., a wafer) at a concentration of between about 0.001% to about 10% by weight, between about 0.001% to about 5% by weight, between about 0.001% to about 1% by weight, between about 0.1% to about 5% by weight, between about 0.5% to about 2% by weight, between about 0.01% to about 10% by weight, between about 0.1% to about 10% by weight or between about 1% to about 10% by weight. In other embodiments, a permeation enhancer may be included in a device (e.g., a wafer) at a concentration of greater than 0.001% by weight, greater than 0.01% by weight, greater than 0.05% by weight, greater than 0.1% by weight, greater than 0.5% by weight, greater than 1% by weight, greater than 2% by weight, greater than 5% by weight, or greater than 10% by weight.

In some cases, the device may be capable of delivering an active agent such that the concentration of the active agent attains a level of between about 1 ng/mL and about 1 mg/mL, in some embodiments between about 10 ng/mL and about 1 mg/mL, in some embodiments between about 100 ng/mL and about 1 mg/mL, in some embodiments between about 1 micrograms/mL and about 1 mg/mL, in some embodiments between about 10 micrograms/mL and about 1 mg/mL, in some embodiments between about 1 ng/mL and about 100 micrograms/mL, in some embodiments between about 1 ng/mL and about 10 micrograms/mL, and in some embodiments between about 1 ng/mL and about 1 microgram/mL.

A device may be manufactured by any suitable method. In certain embodiments, a device may be manufactured under sterile conditions. In other embodiments, a device may be sterilized prior to packaging the device. In certain embodiments, the device may be sterilized prior to administration to a subject. In some embodiments, a device may be manufactured using process comprising salt leaching, solvent casting, molding, spray coating, spray drying, spin coating, and/or compression. Other methods will be known to those of ordinary skill in the art. In some embodiments, a coating may be applied to a device precursor and the coating compressed to form a layer (e.g., a backing layer or a mucoadhesive layer). In some embodiments, a layer (e.g., a mucoadhesive layer, a backing layer, and/or a sacrificial layer) may be applied using a spray-coating process. In some embodiments, a layer mucoadhesive material may be coated on a device by dissolving a layer material in an appropriate solvent (e.g., water) and applying the resultant solution onto the device. The coating may be applied using any suitable technique, such as spraying. Alternatively, a layer may be applied in dry form. For example, solid powder of a layer material may be applied to a device and compressed to form a layer. In some embodiments, the active agent release compartment may be prepared first and the backing layer and any additional layer applied to the active agent release compartment. In other embodiments, two or more components of a device may be prepared and then assembled. For instance, a backing layer shell may be prepared and an active agent release compartment may be placed into the backing layer shell to form a device. The backing layer shell and active agent release compartment may, in some cases, be bonded using any suitable method. For example, in some embodiments a backing layer and an active agent release compartment may be bonded using an adhesive or compression.

In some cases, a device may be manufactured on a surface. For example, an active agent-containing material may be deposited on a surface and one or more coatings may be applied to the active agent-containing material to create a device. In some instances, the device may be removed from the surface such that the portion of the device that was in contact with the surface is essentially free of the coating. In one non-limiting example, a device may have a first side and a second side (e.g., a front side and a back side). The device may be positioned on a surface such that the first side is in contact with the surface. One or more coatings may then be applied to the device onto the second side. The first side may be shielded from the coating since the first side is in contact with the surface. In some instances, a coating may coat all sides of the device not in contact with the surface. The device may then be removed from the surface. Such a method may be used, for example, to construct a device by successively applying layers to a surface. In some cases, material for manufacturing the device may be deposited in discrete areas of a surface, where a device is manufactured at each discrete area. In other embodiments, surface larger than an individual device may be coated successively, and individual devices may be cut out of the resultant layered construct (e.g., with a hole-punch).

In some embodiments, active agent-containing particles (e.g., microparticles and/or nanoparticles) may be incorporated into a device. Particles may be manufactured using any suitable method. For example, in some embodiments, an active agent may be encapsulated in particles using, for example, spray drying, interfacial polymerization, hot melt encapsulation, phase separation encapsulation, spontaneous emulsion, solvent evaporation microencapsulation, salvent removal microencapsulation, coacervation, and low temperature microsphere forrnation.

Also described herein are systems for delivery of mucoadhesive devices, for example, for delivery of a plurality of devices.

In some embodiments, a system may comprise one or more mucoadhesive devices (e.g., wafers) configured for release of an active agent. The system may, in some embodiments, comprise a plurality of devices encapsulated in a containment vehicle. For instance, the plurality of devices may be encapsulated in a capsule, a caplet, a gelcap, or a tablet. The containment vehicle may be configured to release one or more devices in a desired location, e.g., the intestine.

In some embodiments, system may comprise a plurality of devices encapsulated in a containment vehicle. For instance, the plurality of devices may be encapsulated in a capsule, a caplet, a gelcap, or a tablet. The containment vehicle may be configured, in some embodiments, to dissolve in certain regions of a subject (e.g., the small intestine or the large intestine) and/or under certain conditions (e.g., within certain pH ranges). For example, the containment vehicle may be enteric coated. An enteric coating may be any suitable coating that allows the containment vehicle to release the devices in the small intestine. In sonic cases, an enteric coating may dissolve preferentially in the small intestine as compared to the stomach. In other embodiments, the enteric coating may hydrolyze preferentially in the small intestine as compared to the stomach. Non-limiting examples of materials used as enteric coatings include methyl acrylate-methacrylic acid copolymers, cellulose acetate succinate, hydroxy propyl methyl cellulose phthalate, hydroxy propyl methyl cellulose acetate succinate (i.e., hypromellose acetate succinate), polyvinyl acetate phthalate (PVAP), methyl methacrylate-methacrylic acid copolymers, and sodium alginate and stearic acid. Other examples and embodiments are discussed in more detail elsewhere herein.

An enteric coating may also be applied to the devices described herein (such as a wafer), regardless whether the containment vehicle is enteric coated or not. For example, if the containment vehicle is not enteric coated, and the device(s) within the containment vehicle is (are) enteric coated, the containment vehicle may preferentially dissolve in the stomach, allowing the device(s) (such as wafer(s)) within the containment vehicle to be released; and the enteric coating of the device(s) allows the device(s) to dissolve preferentially in the small intestine as compared to the stomach. Alternatively, the containment vehicle may also be enteric coated, such that the containment vehicle preferentially dissolves in the intestine, and the enteric coating layer on the device(s) further protects the device(s) until the layer is dissolved.

In some instances, the containment vehicle may comprise between 2 and 9 devices, in some embodiments between 11 and 15 devices, and in some embodiments, between 16 and 20 devices. In certain embodiments, the containment vehicle may contain 2, 3, 4, 5, 6, 7, 8, or 9 devices.

In some embodiments, a device may be sufficiently flexible to be rolled and placed within a containment vehicle. In some instances, a large device may be rolled into a smaller configuration and placed into a containment vehicle suitable for oral administration. The device may be released in the subject (e.g., in the gastrointestinal tract) where the device may unroll and adhere to a wall of the gastrointestinal tract (e.g., a mucosa). As discussed elsewhere herein, the device may comprise one or more additives (e.g., plasticizers) that improve the flexibility of the device.

In some embodiments, the device may be configured such that the device does not substantially adhere to (i.e., aggregate with) one or more other devices. For example, the device may comprise an anti-adhesion agent that substantially reduces the adhesion of one device for another. In some embodiments, the anti-adhesion agent may be a layer on the device. In some cases, the layer may at least partially coat the attachment region. In some embodiments, the layer may substantially coat the entire attachment region. In certain embodiments, the layer may substantially coat the entire device, In some embodiments, the anti-adhesive layer may dissolve or degrade over a short period of time to allow the devices to drift away from each other. For example, the anti-adhesion layer may be configured to dissolve or degrade over a period of between about 1 minute and about 180 minutes, in some embodiments between about 1 minute and about 120 minutes, in some embodiments between about 1 minute and about 60 minutes, in some embodiments between about 1 minute and about 30 minutes, in some embodiments between about 10 minutes and about 120 minutes, in some embodiments between about 20 minutes and about 120 minutes, and in some embodiments between about 30 minutes and about 120 minutes. In some embodiments, the anti-adhesive agent may be prepared from, for example, sugars, polymers, proteins, or other molecules. In a non-limiting example, the anti-adhesion agent may be a polyalkylene glycol (e.g., polyethylene glycol), silica, and/or magnesium stearate. In certain embodiments, the anti-adhesive layer may comprise a dispersal agent (e.g., a disintegrant). For example, the disintegrant may be an expandable polymer. Non-limiting examples of disintegrants include polymers such as crosslinked polyvinylpyrrolidone (crospovidone), crosslinked sodium carboxymethyl cellulose (croscarmellose sodium), and sodium starch glycolate.

In some embodiments, the anti-adhesion layer or anti-adhesion agent may emit a gas. For example, the anti-adhesion layer or anti-adhesion agent may effervesce upon contact with an aqueous environment. In some cases, release of a gas may facilitate separation of the devices (e.g., wafers). An anti-adhesion layer or anti-adhesion agent that emits a gas may, in some embodiments, comprise a dispersal agent that include a combination of a carbonate and an acid that may react to produce carbon dioxide gas upon contact with an aqueous solution. For example, in some cases, the carbonate may be a bicarbonate. Non-limiting examples of the carbonate counter ions or bicarbonate counter ion are sodium, potassium, magnesium, and calcium. The acid may be any biocompatible acid capable of reacting with the carbonate to release a gas (e.g., carbon dioxide). In some cases, the acid may be a non-volatile acid. Non-limiting examples of acids include citric acid, ascorbic acid, lactic acid, and glycolic acid. The carbonate and acid may be present in any ratio suitable to produce effervescence. In some embodiments, the carbonate and acid may be present as a mixture to form an anti-adhesive agent, or as a mixture in the anti-adhesion layer. In other embodiments, the carbonate and acid may be in isolated in separate regions of an anti-adhesion layer.

In certain embodiments, the adhesiveness of one device for another device may be reduced or substantially eliminated by the geometrical configuration of the device. For example, in some embodiments, the device may have a non-planar shape, which assists in minimizing aggregation of the device. In some instances, the device may have configured as a hemisphere. In other embodiments, the device may be configured as a cylinder, a rod, an ellipsoid, or a sphere, a doughnut, a toroid, a pyramid, a triangle, a star shape, an irregular shape. and the like.

In some cases, a plurality of devices may be placed and delivered within a dissolvable container which is under slight over-pressure. Upon dissolution of the container, the over-pressure pushes the devices away from each other, thereby minimizing self-aggregation.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal including mammals, preferably mice, rats, other rodents, rabbits, dogs, cats, swine, cattle, sheep, horses, or primates, and most preferably humans. The compounds can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism. Veternary animals are contemplated herein and include birds (e.g., domestic fowl) and reptiles (e.g., snakes).

In the present specification, the term “therapeutically effective amount” means the amount of the subject compound that will elicit the biological or medical response of a tissue, system, animal, or human that is being sought by the researcher, veterinarian, medical doctor, or other clinician. The compounds are administered in therapeutically effective amounts to treat a disease. Alternatively, a therapeutically effective amount of a compound is the quantity required to achieve a desired therapeutic and/or prophylactic effect, such as regulation of blood glucose levels.

In some embodiments, a device may be administered to a subject. In some cases, the device may be administered as a single device. In other embodiments, a plurality of devices may be administered. As described herein, in some embodiments, a device may be administered in a containment vehicle. It should be understood that the device may be an isolated device or may be a member of a plurality of devices.

In certain embodiments, adhesion of a device to a tissue (e.g., a mucosa) in the gastrointestinal system of a subject may be facilitated by peristalsis. Thus, in some embodiments, a device may be administered with a peristalsis enhancer. Non-limiting examples of peristalsis enhancers include magnesium (Mg) salts, senna, fiber, bisacodyl, and the like, and combinations thereof. In some cases, the peristalsis enhancer may be incorporated into the device and/or system. In some instances, the peristalsis enhancer may be administered separately, i.e., concurrently with, prior to, or after administration of the device. In some embodiments, a peristaltic reflex of a subject may be leveraged. Thus, in certain embodiments, the device may be administered concurrently with, prior to, or after consumption of food by a subject. In some embodiments, the device may be administered at least about 5 minutes, in some embodiments at least about 10 minutes, in some embodiments at least about 15 minutes, in some embodiments at least about 20 minutes, in some embodiments at least about 30 minutes, or in some embodiments at least about 60 minutes after consumption of food by a subject. In some embodiments, the device may be administered between about 30 minutes and about 120 minutes, in some embodiments between about 30 minutes and about 60 minutes after consumption of food by a subject.

Alternatively, the devices and systems described herein can be administered to a subject in need thereof without food or under a fasting condition. For example, the device may be administered at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, between about 3 hours to about 12 hours, between about 4 hours to about 12 hours, between about 4 hours to about 10 hours, between about 4 hours to about 8 hours, or between about 4 hours to about 6 hours, after consumption of food by a subject.

In certain embodiments, the device or system may be used to deliver insulin to a subject in need thereof. In some embodiments, the device or system may be capable of delivering an active agent (e.g., insulin) such that the blood glucose concentration of a subject may be maintained within a range of 3 mmol/L to 8 mmol/L for at least about 1 hour, in some embodiments at least about 2 hours, in some embodiments at least about 4 hours, in some embodiments at least about 8 hours, in some embodiments at least about 10 hours, in some embodiments at least about 12 hours, in some embodiments at least about 16 hours, in some embodiments at least about 20 hours, in some embodiments at least about 24 hours, in some embodiments at least about 30 hours, in some embodiments at least about 36 hours, and in some embodiments at least about 48 _{hours.}

In certain embodiments, the device or system may be used to deliver calcitonin to a subject in need thereof. For example, the device or system may be used to treat hypercalcemia. In another example, the device or system may be used to treat a bone disease, such as osteoporosis. In yet another embodiment, the device or system may be used to treat a mental disorder, such as bipolar disorder or mania. In some embodiments, the device or system may be capable of delivering an active agent (e.g., calcitonin) such that the plasma calcium concentration of a subject may be reduced after a period of about 1 hour by about 5% to about 50%, in some embodithents about 5% to about 25%, in some embodiments about 10% to about 25%, in some embodiments about 5% to about 10%, in some embodiments about 10% to about 50%, in some embodiments about 15% to about 50%, in some embodiments about 25% to about 50%, and in some embodiments about 30% to about 50%, as compared to the plasma calcium concentration of the subject as measured prior to treatment. In certain embodiments, the reduction in plasma calcium concentration may persist for a period of at least about 1 hour, in some embodiments at least about 2 hours, in some embodiments at least about 4 hours, in some embodiments at least about 8 hours, in some embodiments at least about 10 hours, in some embodiments at least about 12 hours, in some embodiments at least about 16 hours, in some embodiments at least about 20 hours, in some embodiments at least about 24 hours, in some embodiments at least about 30 hours, in some embodiments at least about 36 hours, and in some embodiments at least about 48 hours.

The devices and systems described herein may be used to administer an active agent to patients (animals and humans) in need of such treatment in dosages that will provide optimal pharmaceutical efficacy. It will be appreciated that the number and/or type of devices or systems required for use in any particular application will vary from patient to patient, not only with the particular active agent selected, but also with the concentration of actie agent in the devices, the route of administration (e.g., oral, nasal, vaginal, rectal, and the like), the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases, a compound (i.e., active agent) may be administered, for example, orally, parenterally, by inhalation spray, or rectally in dosage unit formulations containing conventional non-toxic pharmaceutically acceptable carriers, adjuvants, and vehicles. Parenteral administration may include subcutaneous injections, intravenous or intramuscular injections, or infusion techniques.

Treatment can be continued for as long or as short a period as desired. The devices or systems may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved. A treatment regimen can include a corrective phase, during which dose sufficient, for example, to reduce symptoms is administered, and can be followed by a maintenance phase, during which a lower dose sufficient to maintain the reduced symptoms is administered. A suitable maintenance dose is likely to be found in the lower parts of the dose ranges provided herein, but corrective and maintenance doses can readily be established for individual subjects by those of skill in the art without undue experimentation, based on the disclosure herein.

In another aspect, devices contemplated here may be formulated together with a pharmaceutically acceptable carrier. In particular, the present disclosure provides devices formulated together with one or more pharmaceutically acceptable carriers. The most suitable form of administration in any given case will depend on the degree and severity of the condition being treated and on the nature of the particular active agent being used. For example, the devices may be formulated as a unit dose and/or may be formulated for oral administration.

Exemplary devices may be used in the form of a pharmaceutical preparation, for example, in solid, semisolid, or liquid form, which contains the devices, in admixture with an organic or inorganic carrier or excipient suitable for external, enteral, or parenteral applications. The devices may be combined, for example, with the usual non-toxic, pharmaceutically acceptable carriers for tablets, pellets, capsules, suppositories, solutions, emulsions, suspensions, and any other form suitable for use. As the devices can vary in size from, for example, nanoscale dimensions to millimeters or even larger, the size of the devices may be considered when contemplating a formulation of the devices. For example, the devices may be prepared as a suspension in a liquid formulation. In other instances, the devices may be trapped in a gel formulation. In still other examples, the devices may be contained in a solid formulation, such as a capsule. The active agent may be included in the devices in an amount sufficient to produce the desired effect upon the process or condition of the disease.

For preparing solid formulations of devices such as tablets, the devices may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a heterogeneous mixture of devices and one or more carriers. When referring to these preformulation compositions as homogeneous, it is meant that the devices are dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the devices may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid: (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the devices moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

The devices may, in some embodiments, be formulated for inhalation or insufflation and include solutions and suspensions in pharmaceutically acceptable, aqueous or organic solvents, or mixtures thereof, and powders, provided that they have dimensions suitable for inhalation or insufflation. Such formulations may be advantageous, for example, for delivery of devices to a mucosa in the nasal passage or sinuses.

Liquid dosage forms for oral administration of the devices include pharmaceutically acceptable emulsions, microemulsions, solutions, suspensions, syrups and elixirs. In addition to the devices, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof. In certain embodiments, the devices may comprises a coating that substantially inhibits release of the active agent while the devices are in a liquid dosage formulation. In other embodiments, the liquid dosage formulations may be formulated to substantially prevent release of the active agent from the devices. For example, the liquid formulation may have a pH range that differs from the pH range in which active agent is released from the devices.

Suspension of devices may be facilitated by addition of suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing devices with one or more suitable non-irritating excipients or carriers comprising, for example, cocoa butter, polyethylene glycol, a suppository wax or, a salicylatc, and which is solid at room temperature, but liquid at body temperature and, therefore, will melt in the body cavity and release the devices.

Ointments, pastes, creams and gels may contain, in addition to devices, excipients, such as animal and vegetable fats, oils, waxes, paraffins, starch, tragacanth, cellulose derivatives, polyethylene lycols, silicones, bentonites, silicic acid, talc and zinc oxide, or mixtures thereof.

Powders and sprays may contain, in addition to devices, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Pharmaceutical compositions suitable for parenteral administration comprise devices in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, hacteriostats, solutes which render the formulation isotonic with the blood of the intended recipient or suspending or thickening agents. Parenteral administration of the devices may allow the devices to adhere to the gut mucosa and deliver an active agent.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required device size in the case of dispersions, and by the use of surfactants.

In another aspect, enteral pharmaceutical formulations including devices, an enteric material; and a pharmaceutically acceptable carrier or excipient thereof are provided. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4,of about 6.6, of about 6.8, of about 7.0, of about 7.2, of about 7.4, of about 7.6, of about 7.8,of about 8.0, of about 8.2, of about 8.4, of about 8.6, of about 8.8, of about 9.0, of about 12, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and malcic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that may be used.

Advantageously, kits are provided for use by, for example, a diabetic in need of blood glucose concentration control. Such kits include a suitable dosage form of devices such as those described above and instructions describing the method of using such devices to control blood glucose concentration. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like, comprising devices).

### EXAMPLES

### Example 1

### Release of Sulforhodamine B

Wafers were prepared using a mixture of Carbopol 934, pectin and sodium carboxylmethylcellulose with a Carbopol/pectin/SCMC weight ratio of 1:1:2. Suforhodamine B was added to this mixture such that the final concentration in each wafer was 3-5% w/w. The mixture was compressed under a pressure of 1 or 2 tons using a hydraulic press. A hole punch was used to cut this disk into smaller disks with radii of 5 mm. These disks were placed on a support and coated on all sides but one using a solution of 5% w/v ethylcellulose in acetone. Acetone was evaporated at room temperature. The wafers were placed in a solution of HBSS and amount of sulforhodamine released at various times was measured.

The results are shown in FIG. 2 and indicate that linear release of the sulforhodamine occurred over a period of 3-4 hours.

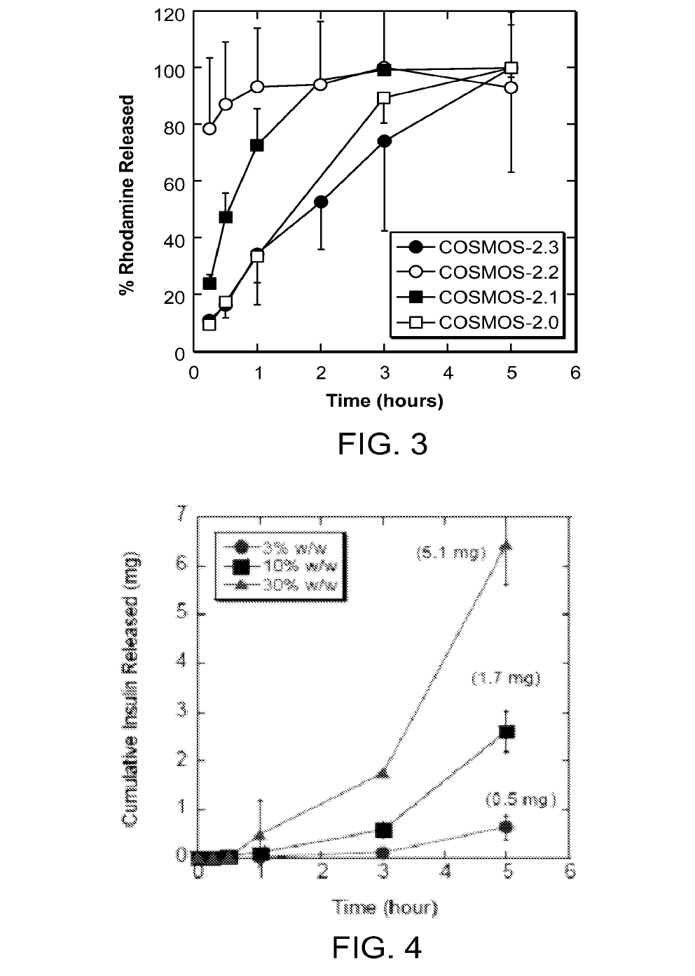

FIG. 3 shows that the release kinetics of sulforhodamine could be adjusted by different formulations.

### Example 2

### Release of Insulin

Wafers were prepared using a mixture of Carbopol 934, pectin and sodium carboxylmethyleellulose with a Carbopol/pectin/SCMC weight ratio of 1:1:2. FITC-labeled bovine insulin was added to this mixture such that the final concentration in each wafer was 3-30% w/w. The mixture was compressed using a hydraulic press. A hole punch was used to cut this disk into smaller disks with radii of 5 mm. These disks were placed on a support and coated on all sides but one using a solution of 5% w/v ethylcellulose in acetone. Acetone was evaporated at room temperature. The wafers were placed in a solution of HBSS and amount of FITC-insulin released at various times was measured.

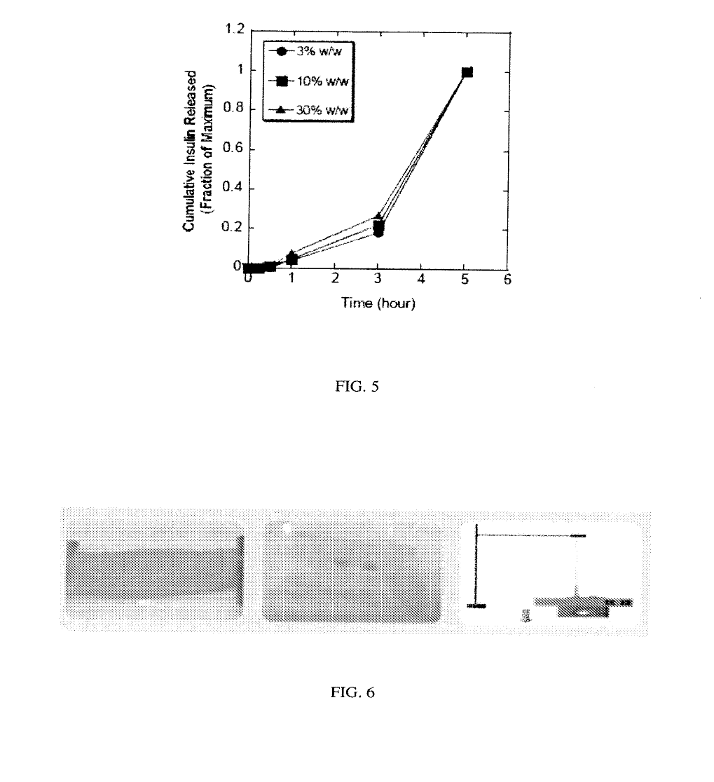

The results are shown in FIG. 4 and indicate that insulin release was dose-proportional. Normalized data from FIG. 4 are presented in FIG. 5, which shows that the kinetics of insulin release were dose-independent and complete in about 5 hours.

### Example 3

### Adhesion of Wafers

Experiments were performed on wafers generated using the methods of Example 2 to determine the adhesion force between the wafer and the intestine. Pig intestine was used in these studies. The intestine was rinsed with PBS and then cut into 5 cm long loops. One end of the loop was ticd off HBSS was added to the lumen. Wafers were randomly inserted in the intestine sections. The other end of the section was also tied off. The whole intestinal section was placed on a rocker and was shaken for various times, typically 30 minutes. The intestine was longitudinally dissected and the mucosal surface (where the wafers were still attached) was mounted onto a microbalance using clamps to secure it. A small piece of a plastic cylinder (2 cm in length and 1 mm in diameter) was super glued to the backing side of one of the patches on the mucosa. The other end of the cylinder was attached to a string and passed over a pulley. The cylinder was gradually pulled until the patch detached from the mucosa. The detachment force (force of adhesion) at which the adhesive bond between the patch and the mucosa failed was recorded.

FIG. 6 shows a photograph of the clamped intestine (left panel), a photograph of the opened intestine with the wafers attached to the wall of the intestine on the mucoadhesive side of the wafers (center panel), and a schematic of the technique used to measure the adhesion force of the wafers (right panel).

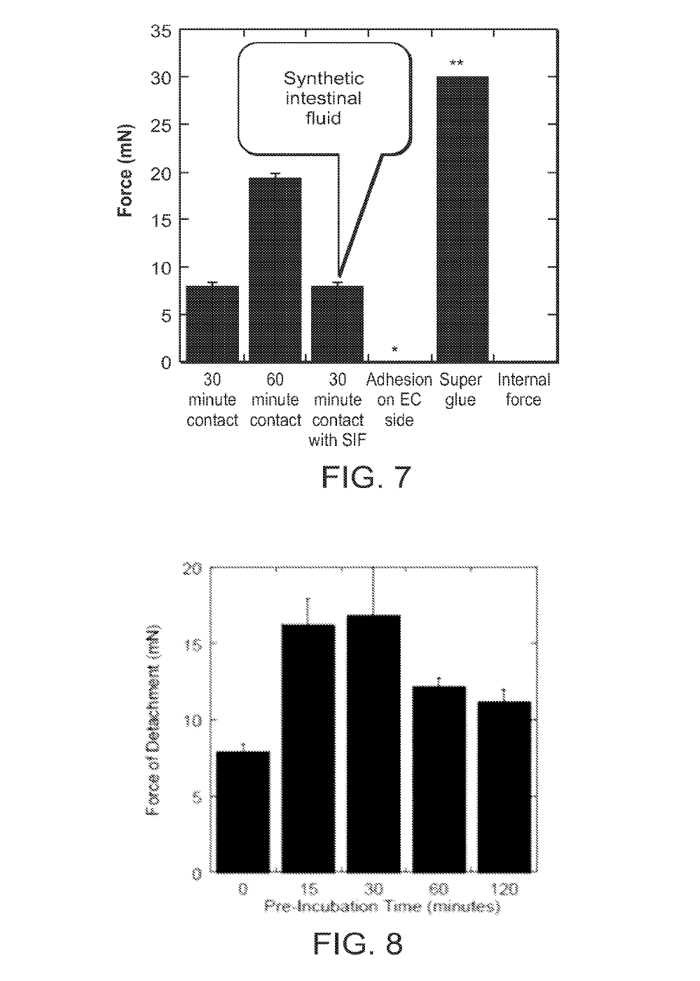

FIG. 7 shows the results of the adhesion experiments and demonstrate that the wafers adhered with a force greater than 40 times their weight, that the force of adhesion was higher after 60 minutes of contact with the intestine as compared to 30 minutes, that wafers adhered well to intestinal fluid, and that substantial adhesive force was only produced on the mucoadhesive side of the wafers.

FIG. 8 shows that preincubation of the wafers in HBSS improved the adhesive properties of the wafers.

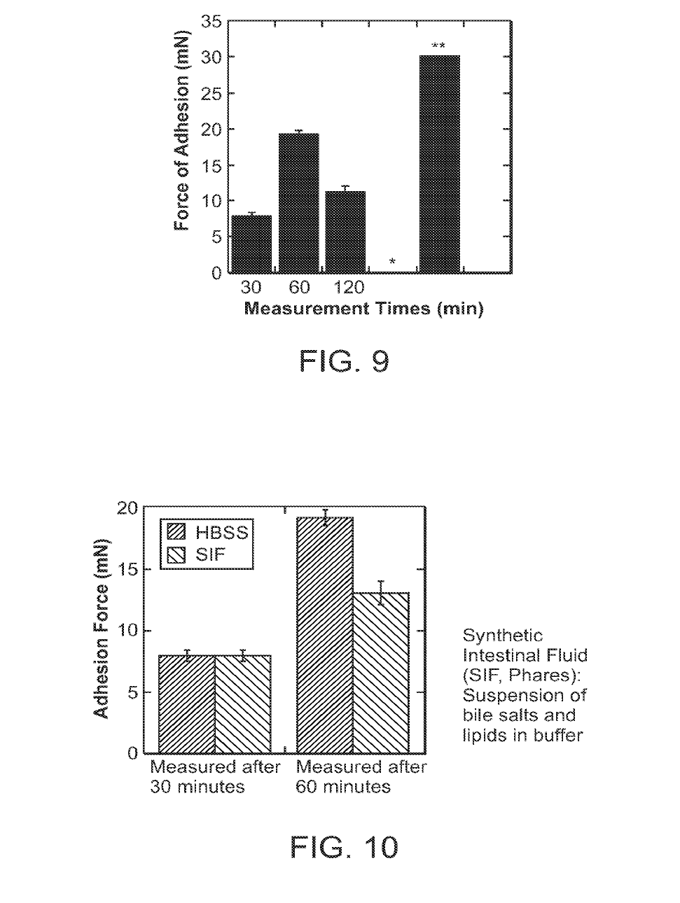

FIG. 9 shows the contact time dependence of adhesion force between the wafer and the intestine and demonstrates that the adhesion force decreased between 60 minutes and 120 minutes of contact time.

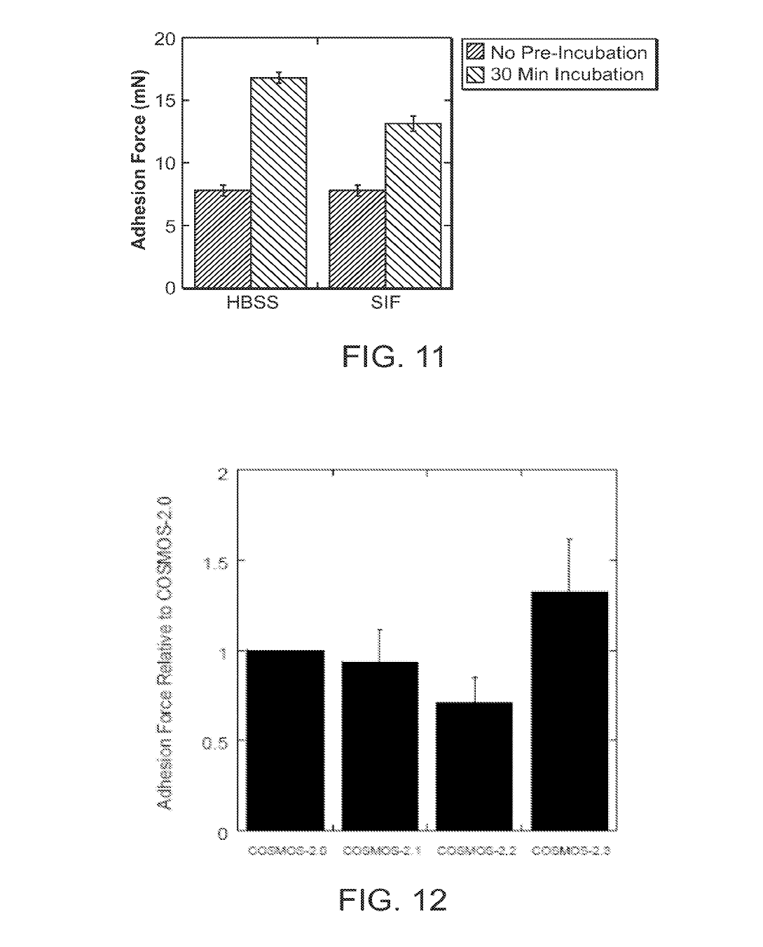

FIG. 10 shows that adhesion strenath increased over time and that the increase was more prominent with HBSS as compared to synthetic intestinal fluid (SIF).

FIG. 11 shows that preincubation of wafers in HBSS or SIF increased adhesion strength and that the increase was more prominent with HBSS as compared to SIF.

FIG. 12 shows the adhesion force of four different wafer formulations and demonstrates that the formulation of the wafers can be adjusted to modify the adhesion force.

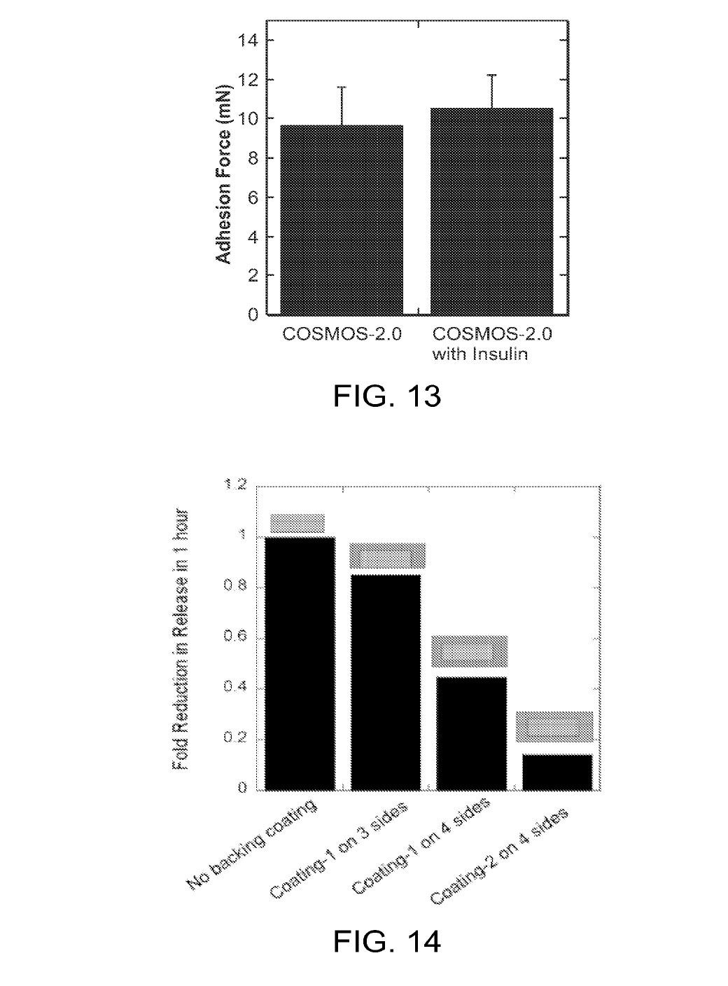

FIG. 13 shows the adhesion strength for a wafer without insulin and a wafer with a 30% loading of insulin. The results demonstrate that the presence of insulin in the formulation did not significantly affect the adhesion force.

### Example 4

### Impact of Backing Layer

Wafers were prepared using a mixture of Carbopol 934, pectin and sodium carboxylmethyleellulose with a Carbopol/pectin/SCMC weight ratio of 1:1:2. The mixture was compressed under a pressure of 1 or 2 tons using a hydraulic press. A hole punch was used to cut this disk into smaller disks with radii of 5 mm. These disks were placed on a support and coated on all sides but one using a solution of 5% w/v ethylcellulose in acetone. In some experiments, the wafer was coated on all sides by EC. In one experiment (Coating-2) EC was replaced by PLGA. Acetone was evaporated at room temperature. The wafers were placed in a solution of HBSS and amount of sulforhodamine released at various times was measured.

FIG. 14 shows the impact of the backing layer on the release of an active agent and demonstrate that the backing layer enabled control over the release rate of the active agent.

### Example 5

### Transport of FITC-Insulin Across Caco-2 Monolayer

Caco-2 cell line HTB-37 (American Type Culture Collection, Rockville, Md.), derived from human colon cells, was used for all experiments. Cells were maintained at 37° C. in DMEM supplemented with 25 IU/ml of penicillin, 25 mg/L of streptomycin, 250 μg/L of amphotericin B and 100 ml/L of fetal bovine serum. Monolayers were grown on BD Biocoat™ collagen filter supports (Discovery Labware, Bedford. Mass.). Feeding schedules remained the same for all experiments to ensure comparable monolayer growth. At the end of the growth period, the integrity of the cell monolayer was confirmed by transepithelial electrical resistance (TEER) measurements (Millicell-ERS voltohmmeter. Millipore. Billerica, Mass.). Only monolayers with TEER values over 300 Ωcm² were used for further experimentation. A solution of FITC-insulin in DMEM was placed on Caco-2 monolayers at various concentrations and samples were collected from the basolateral side and analyzed for fluorescence.

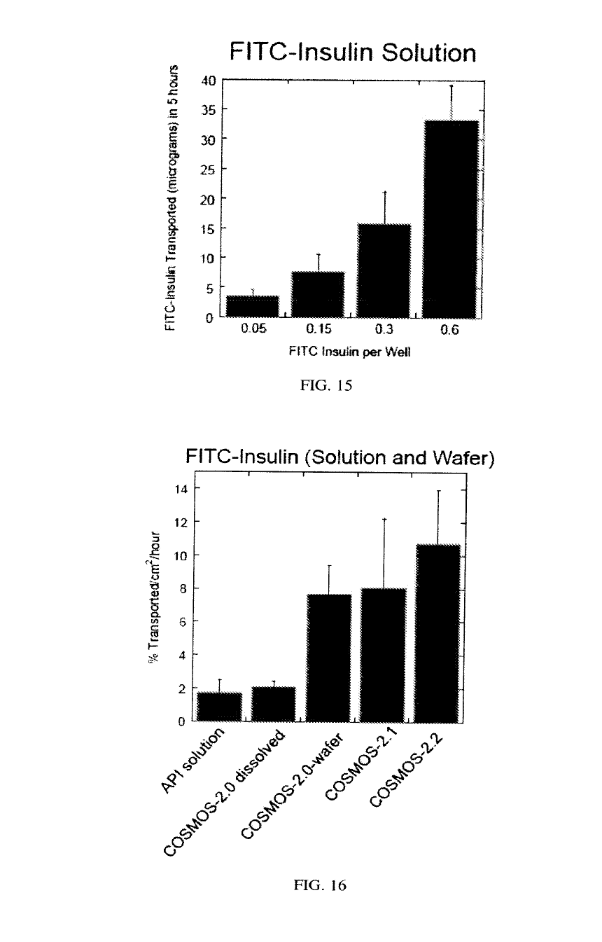

FIG. 15 shows the transport of insulin free in solution across the Caco-2 monolayers was dose-dependent.

To determine insulin transport from wafers, FITC-insulin-containing wafers were placed on Caco-2 monolayers. Samples were taken from the basolateral side and analyzed for insulin concentration. FIG. 16 shows that transport of insulin across the Caco-2 monolayers was substantially higher when released from wafers as compared to when free in solution.

### Example 6

### Transport of Rhodamine Across Caco-2 Monolayer

Caco-2 cell line HTB-37 (American Type Culture Collection, Rockville, Md.), derived from human colon cells, was used for all experiments. Cells were maintained at 37° C. in DMEM supplemented with 25 IU/ml of penicillin, 25 mg/L of streptomycin, 250 ug/L of amphotericin B and 100 ml/L of fetal bovine serum. Monolayers were grown on BD Biocoat™ collagen filter supports (Discovery Labware, Bedford. Mass.). Feeding schedules remained the same for all experiments to ensure comparable monolayer growth. At the end of the growth period, the integrity of the cell monolayer was confirmed by transepithelial electrical resistance (TEER) measurements (Millicell-ERS voltohmmeter, Millipore, Billerica, Mass.). Only monolayers with TEER values over 300 Ωcm² were used for further experimentation. A solution of rhodamine in DMEM was placed on Caco-2 monolayers at various concentrations and samples were collected from the basolateral side and analyzed for fluorescence. To determine rhodamine transport from wafers, rhodamine-containing wafers were placed on Caco-2 monolayers. Samples were taken from the basolateral side and analyzed for rhodamine concentration.

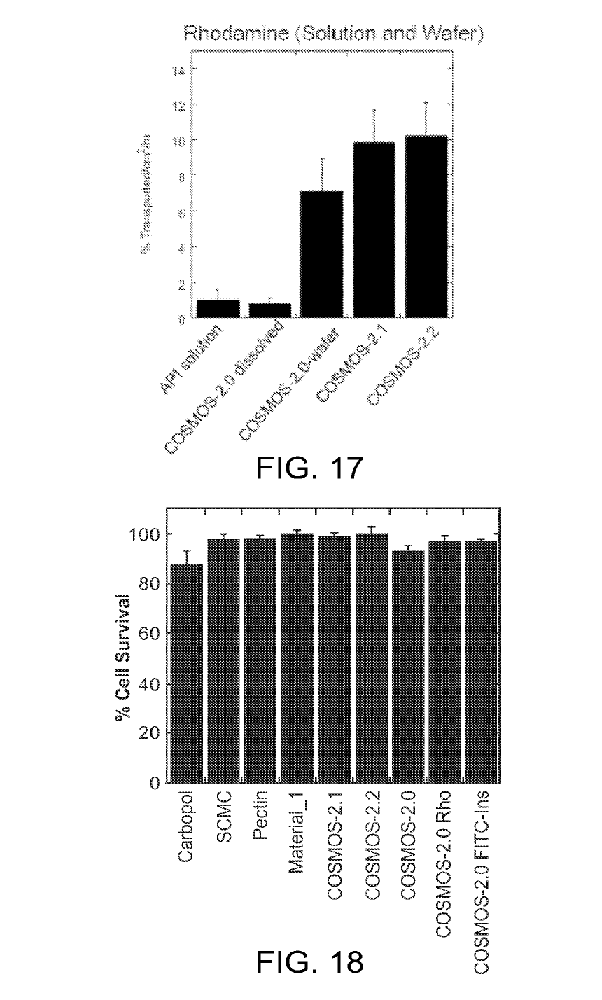

FIG. 17 shows that transport of rhodamine across the Caco-2 monolayers was substantially higher when released from wafers as compared to when free in solution.

### Example 7

### Cytotoxicity of Wafers

Coco-2 cells were seeded at 10⁵ cells/well onto a 96-well plate. Enhancer solutions (100 μl) were applied for 30 min. Ten microliters of reagent from an MTT kit (American Type Culture Collection, Rockville, Md.) was applied to each well for 5 h, after which 100 μl of detergent was applied to each well and allowed to incubate in the dark at room temperature for about 40 h. Absorbance was read at 570 nm (MTT dye) and 650 nm (detergent). Percent survival values are reported as the fraction of viable cells, as compared to the negative control, DMEM.

FIG. 18 shows the results and demonstrates that all of the wafer formulations exhibited minimal cytotoxicity.

### Example 8

### Impact of Release Rate Enhancers and Peptide Drugs

Experiments were performed on wafers generated using similar methods to Example 2. In some experiments additional release-rate control excipicnts were added to block the active agent from binding to the material in the active agent release compartment. Four excipients (including salt, protein, and surfactant) were individually added to each wafer in the amounts indicated (replacing mucoadhesive polymer matrix). These added excipients were included to improve the release of insulin from the device. The wafers were placed in a solution of HBSS PBS (pH 7.4) with continuous shaking at room temperature and amount of FITC-insulin released at various times was measured. In some experiments, FITC-insulin was replaced with a different peptide drug to show the general utility of the devices. In these experiments, the concentration of the peptide drug after dissolution was measured using a commercial ELISA assay.

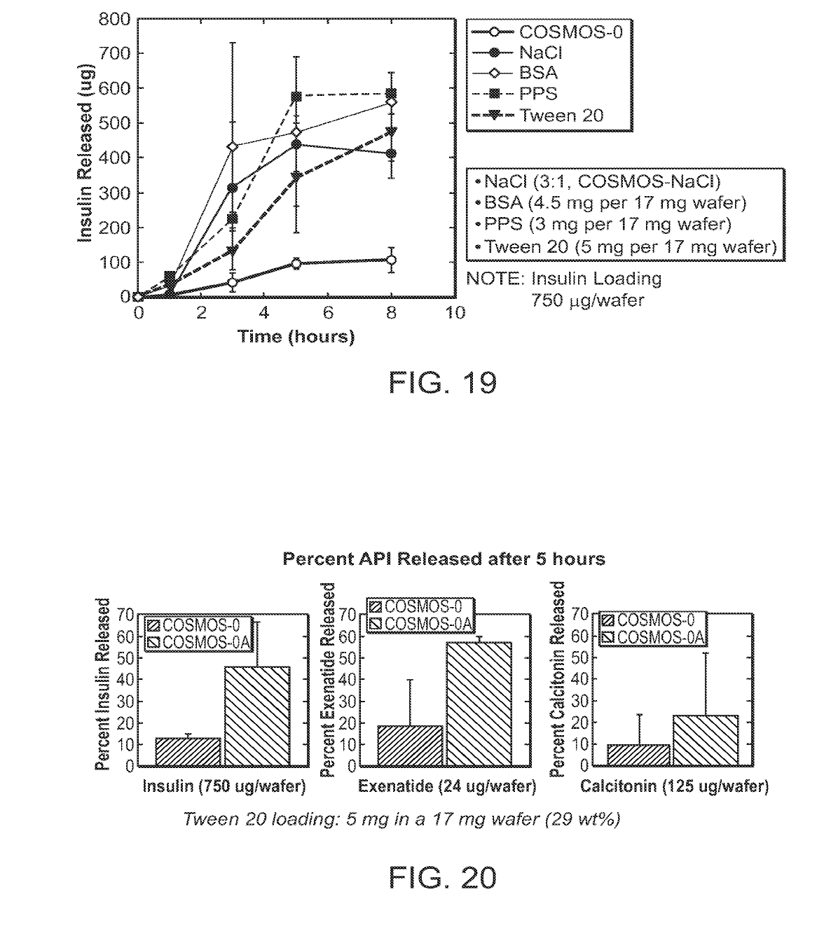

The results are shown in FIG. 19 and indicate that the insulin release rate is highly dependent on the specific excipients chosen to control the release. The inclusion of stable, non-toxic polysorbate surfactant Tween 20 at 29 wt % showed a steady release rate over the duration of the experiment. FIG. 20 shows that the release rate is accelerated due to Tween 20 inclusion for two more peptide dtugs in addition to insulin.

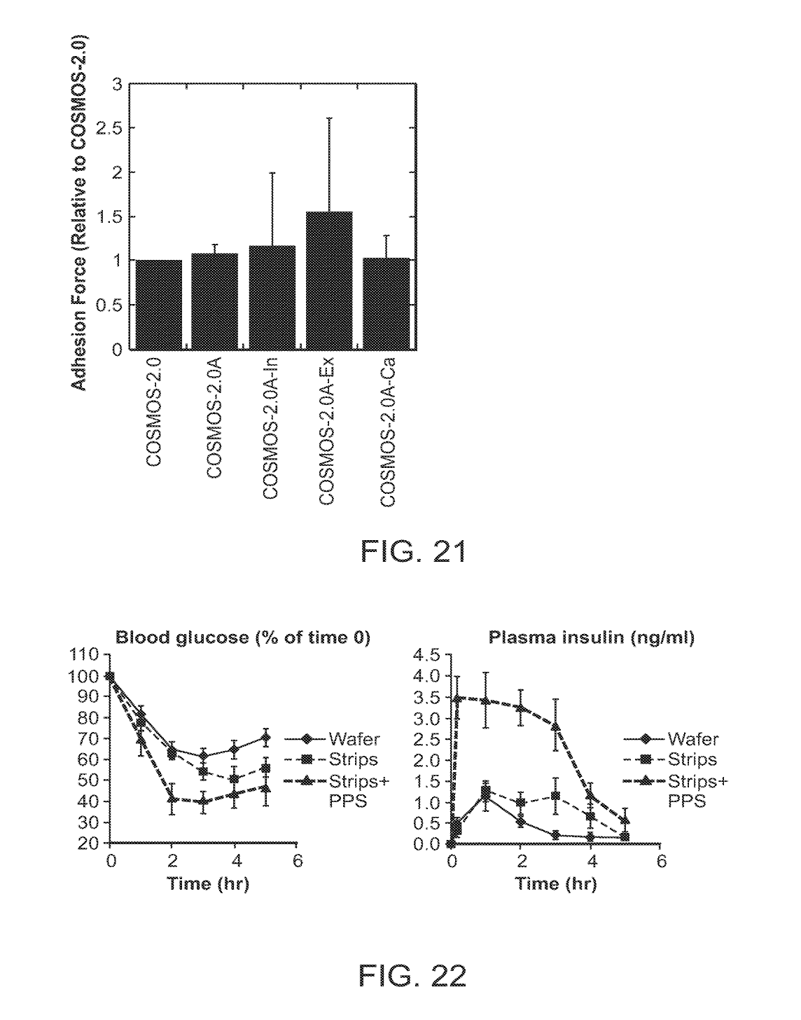

Adhesion forces were measured using the methods of Example 3. The results are shown in FIG. 21 and show that surprisingly, adhesion forces of devices including Tween 20 or different peptides are not significantly different from those without.

### Example 9

### Impact of Geometry and Permeation Enhancers

In vivo pharmacokinetic studies were performed to evaluate the efficacy of devices in enhancing oral bioavailability of insulin. In vivo studies were performed in adult male Sprague-Dawley rats (300-325 g) by surgical administration to the jejunum. Briefly, rats were fasted overnight for 7 hours during regular nocturnal hours before the experiment with free supply to water. During the experiment, rats were anesthetized using isoflurane inhalation, and intestine was exposed by a midline abdominal incision. A small incision (0.3-0.4 cm) was made into the intestinal lumen in the jejunum region of the small intestine (12-15 cm from the stomach), and 3 patches (2 mm diameter) loaded with appropriate dose of bovine insulin were placed into the jejunum. Following the device insertion, the intestinal incision was closed using tissue glue; and 0.5 ml saline was injected into the intestine (15 minutes after device insertion). Blood samples were collected from the animals via the tail vein milking method in heparinized blood collection tubes (no additives added) up to 5 hrs. During collection, blood glucose levels were tested with commercially available blood glucose test strips and devices. Plasma Serum was separated from blood by centrifuging at 5000 rpm for 5 minutes, and was stored at −20° C. until further analysis. Collected plasma serum samples were analyzed by commercially available ELISA kits for insulin concentration.

Cylindrical devices were manufactured by a similar method as Example 2. First, a mixture of bovine insulin, any additional excipients, and a mucoadhesive polymer matrix was compressed. The polymer mix was 1:1:2 Carbopol:pectin:sodium carboxy methylecellulose (SCMC). The compression force used was 3 Tons for 5 minutes using a hydraulic press (Carver Inc, Wabash, Ill.) to produce a 400 μm thick matrix. Devices were manufactured from this matrix by first cutting smaller devices using a scalpel or biopsy punch. Two device geometries were compared in this study: 2 mm diameter cylindrical devices and 2 mm wide rectangular strips. Dosing of insulin in the devices was normalized to provide 50 U/kg in each rat. Once the matrix was compressed, the patch was coated on three sides with ethyl cellulose, the backing layer, by painting a 5% w/v solution of ethylcelluose in acetone. Acetone was evaporated at room temperature. This procedure produced a thin layer of EC of about 50 μm.

The results are shown in FIG. 22. Insulin-loaded devices significantly increased insulin bioavailability, whereas insulin solution injected into the small intestine resulted in a negligible amount of insulin appearing into the circulation (data not shown). In addition, the insulin was pharmacologically active as demonstrated by a decreased blood glucose level over time. The geometry of the wafers was a surprisingly strong variable in determining performance. The same dose of insulin administered by the rectangular strips (placed longitudinally in the intestine) delivered more active insulin to the rat than cylindrical devices. Also, the inclusion of a permeation enhancer (1% Palmityldimethyl ammonio propane sulfonate (PPS) also significantly increased the amount of insulin delivered to the blood.

### Example 10

### Release of Calcitonin From Mucoadhesie Devices

Wafers were prepared using a mixture of Carbopol 934, pectin and sodium carboxylmethylcellulose with a Carbopol/pectin/SCMC weight ratio of 1:1:2, Salmon calcitonin was added to this mixture such that the final amount in each wafer was 24 μg. The mixture was compressed using a hydraulic press. A biopsy hole punch was used to cut this disk into smaller disks with diameter of 5 mm. These disks were placed on a support and coated on all sides but one using a solution of 5% w/v ethylcellulose in acetone. Acetone was evaporated at room temperature. The wafers were placed in a solution of PBS at pH 7.4 and the amount of calcitonin released at various times was measured.

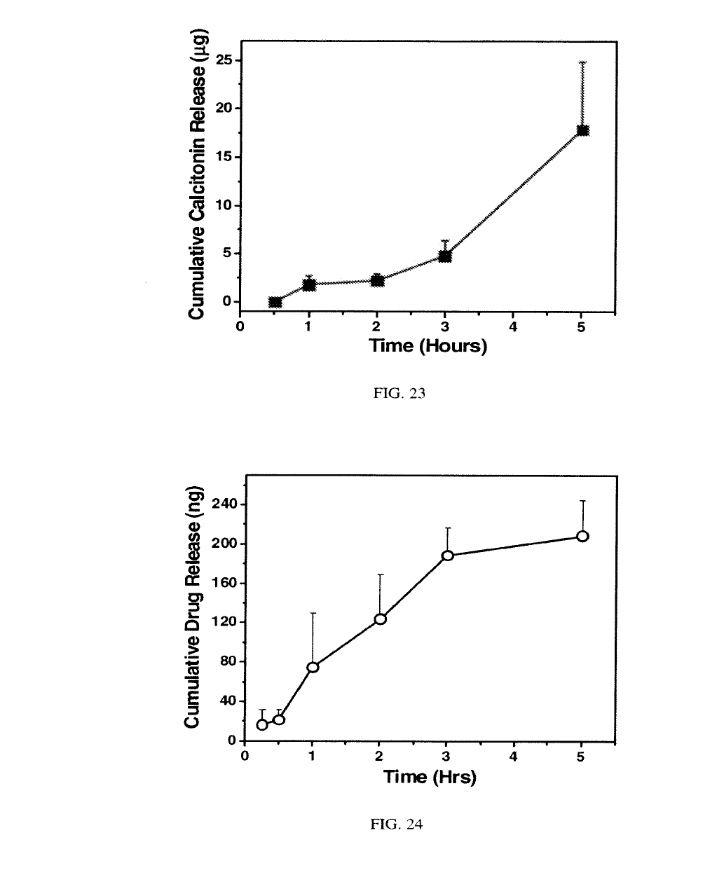

The results are shown in FIG. 23 and indicate that approximately 75% of the loaded calcitonin was detectable in the release medium after 5 hours and that the release kinetics were similar to that of insulin (FIG. 4).

### Example 11

### Transport of Calcitonin Across Caco-2 Monolayer From Mucoadhesive Devices

Caco-2 cell line HTB-37 (American Type Culture Collection, Rockville, Md.), derived from human colon (colorectal adrenocarcinoina) cells, was used for all experiments. Cells were maintained at 37° C. in DMEM supplemented with 25 IU/ml of penicillin, 25 mg/L of streptomycin, and 100 ml/L of fetal bovine serum. Monolayers were grown on BD Biocoat™ HTS collagen filter supports (BD Biosciences, Bedford, Mass.). Feeding schedules remained the same for all experiments to ensure comparable monolayer growth. At the end of the growth period, the integrity of the cell monolayer was confirmed by transepithelial electrical resistance (TEER) measurements (Millicell-ERS voltohmmeter, Millipore, Billerica. Mass.). Only monolayers with TEER values over 150-200 Ωcm² were used for further experimentation. To determine calcitonin transport from wafers, calcitonin-containing wafers were placed on Caco-2 monolyaers. Samples were taken from the basolateral side and analyzed for calcitonin concentration using a commercial ELISA.

FIG. 24 shows the transport of calcitonin across the Caco-2 monolayer, Mucoadhesive devices were able to transport significant amounts of calcitonin through the caco-2 monolayers without damaging the intercellular tight junctions in the monolayer (observed by stable TEER values).

### Example 12

### Transport of Calcitonin Across the Intestine in Live Rats From Mucoadhesive Devices

In vilio pharmacokinetic and pharmacodynamic studies were performed to evaluate the efficacy of devices in enhancing oral bioavailability of calcitonin. In vivo studies were performed in adult male Sprague-Dawley rats (300-325 g) by surgical administration to the duodenum or jejunum. Briefly, rats were fasted overnight for 7 hours during regular nocturnal hours before the experiment with free supply to water. During the experiment, rats were anesthetized using isotlurane inhalation (for example, at 2.0-2.5% or 4%), and intestine was exposed by a midline abdominal incision. A small incision (0.3-0.4 cm) was made into the intestinal lumen in either the duodenarn region of the small intestine (5-10 cm from the stomach) or the jejunum region of the small intestine (12-15 cm from stomach), and 3 patches (2 mm diameter) loaded with appropriate dose of calcitonin were placed into the intestine. Following the device insertion, the intestinal incision was closed using tissue glue; and 0.5 ml saline was injected into the intestine (15 minutes after device insertion). Control groups included subcutaneous injection after sham surgery and intestinal injection of calcitonin in solution after sham surgery. Blood samples were collected from the animals via the tail vein milking method in heparinized blood collection tubes up to 5 hrs. Plasma was separated from blood by centrifuging at 5000 rpm for 5 minutes, and was stored at −20° C. until further analysis. Collected plasma samples were analyzed by commercially available ELISA kits for calcitonin concentration and by commercially available colorimetric kits for calcium concentration.

Cylindrical devices were manufactured by a similar method as Example 10. First, a mixture of calcitonin, any additional excipients, and a mucoadhesive polymer matrix was compressed. The polymer mix was 1:1:2 Carbopol:pectin:sodium carboxy methylecellulose (SCMC). The compression force used was about 3 Tons (i.e., 2-3.5 Tons) for 5 minutes using a hydraulic press (Carver Inc, Wabash, Ill.) to produce a ˜400 μm thick matrix. Devices were manufactured from this matrix by first cutting smaller devices using a 2 mm biopsy punch. Dosing of calcitonin in the devices was normalized to provide 3 mg/kg in each rat. Once the matrix was compressed, the patch was coated on three sides with ethyl cellulose, the backing layer, by painting a 5% w/v solution of ethylcelluose in acetone. Acetone was evaporated at room temperature. This procedure produced a thin layer of EC of about 50 μm (or for example, 25-100 μm).

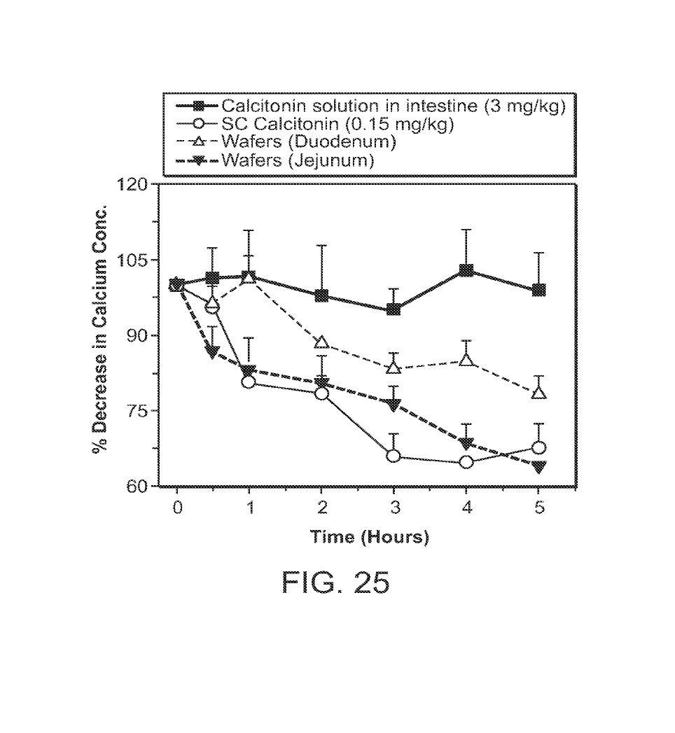

The mucoadhesive devices delivered active calcitonin through the intestine into the blood. The results are shown in FIG. 25. Calcitonin-loaded devices significantly increased pharmacokinetic calcitonin bioavailability, whereas calcitonin solution injected into the small intestine resulted in a negligible amount of calcitonin appearing into the circulation (data not shown). In addition, the calcitonin was pharmacologically active as demonstrated by a decreased plasma calcium level over time (FIG. 25). A significant decrease in plasma calcium concentration was observed with mucoadhesive devices (22% for duodenum, and 36% for jejunum placement) as compared to virtually no decrease with intestinal calcitonin solution.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### Equivalents

While specific embodiments have been discussed, the above specification is illustrative and not restrictive. Many variations will become apparent to those skilled in the art upon review of this specification. The full scope of the embodiments should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtaincd.

What is claimed is:

1. A polymeric controlled release preparation, comprising:
a mucoadhesive layer comprising a first region and a second region, the first region being substantially surrounded by the second region;
wherein
the first region comprises an active agent; and
the second region comprises a mucoadhesive material.

2. The polymeric controlled release preparation of claim 1, wherein the first region comprises polymeric microspheres comprising the active agent.

3. The polymeric controlled release preparation of claim 1, wherein the first region is spheroidal.

4. The polymeric controlled release preparation of claim 1, wherein the first region is an ellipsoid.

5. The polymeric controlled release preparation of claim 1, wherein the first region is a cylinder.

6. (canceled)

7. The polymeric controlled release preparation of claim 1, wherein the preparation has a shape selected from the group consisting of round, ellipsoid, oval, triangular, quadrangular polygonal, and irregular rounded.

8. (canceled)

9. A pharmaceutically acceptable polymeric controlled release device for oral drug delivery, comprising:
a mucoadhesive coating; and
a polymeric release layer comprising an active agent, wherein the polymeric release layer is disposed on the mucoadhesive coating; and wherein the mucoadhesive coating is capable of adhering to a mucosa with a force between 2 and 30 times the weight of the device.

10. The device of claim 9, wherein the mucoadhesive coating is capable of adhering to a mucosa with a force between 2 and 20 times the weight of the device.

11. The device of claim 9, wherein the mucoadhesive coating is capable of adhering to a mucosa with a force between 50 and 100 times the weight of the device.

12.-24. (canceled)

25. A polymeric controlled release device, comprising:
an active agent,
a mucoadhesive layer, and
a permeation enhancer, wherein when a surface of the device adheres to a mucosa defining a privileged region, a substantial majority of the permeation enhancer is maintained within the privileged region.

26. The device of claim 25, wherein substantially none of the permeation enhancer is capable of escaping the privileged region.

27. The device of claim 25, wherein when the surface of the device adheres to the mucosa, molecules that reside outside of the region defined by the surface of the device adhered to the mucosa do not substantially enter the region.

28. The device of claim 25, wherein the device does not substantially enhance permeation of molecules that reside outside the region defined by the surface of the device adhered to the mucosa.

29. The device of claim 25, further comprising polymeric microparticles comprising the active agent.

30.-145. (canceled)
